(19) United States
(12) Patent Application Publication
(10) Pub. No.: US 2025/0107767 A1
(43) Pub. Date: Apr. 3, 2025

(54) SYSTEM AND METHOD FOR ANGIOGRAPHIC DOSE REDUCTION USING MACHINE LEARNING WITH A CONCORDANCE METRIC

(71) Applicants: William E. BUTLER, Bristol, RI (US); AngioWave Imaging, Inc., Concord, MA (US)

(72) Inventors: William E. BUTLER, Bristol, RI (US); Hector ANDRADE-LOARCA, Munich (DE)

(21) Appl. No.: 18/899,118

(22) Filed: Sep. 27, 2024

Related U.S. Application Data
(60) Provisional application No. 63/586,141, filed on Sep. 28, 2023.

(51) Int. Cl. A61B 6/50; A61B 6/00; G06T 7/00; G06T 7/10

(52) U.S. Cl. CPC A61B 6/504; A61B 6/481; G06T 7/0012; G06T 7/10; G06T 2207/20081

(57) ABSTRACT

Methods, systems, and computer readable media are provided for reduced-dose angiography using machine learning (e.g., deep learning) with a concordance metric indicating a degree of similarity between an estimated segmentation of angiographic images generated by a machine learning model and segmented benchmark angiographic images provided as inputs to the machine learning model. Briefly, techniques described herein may use a neural network trained to conserve/preserve angiographic image quality while reducing the angiographic dose of potentially harmful chemical contrast and/or x-ray radiation by employing a concordance metric.

### CROSS-REFERENCE TO RELATED APPLICATIONS

This application claims priority to U.S. Provisional Patent Application No. 63/586,141, filed on Sep. 28, 2023, the disclosure of which is incorporated by reference herein in its entirety.

### FIELD

The present disclosure relates generally to angiography, and more specifically to a system and method for performing angiography with a reduced dosage of chemical contrast agent or x-ray radiation using machine learning.

### BACKGROUND

The heart sends blood as a sequence of arterial stroke volumes throughout the body, where the blood crosses the capillaries to the veins and returns to the heart. The presence and motions of blood in the blood vessels (generally, branching tubular structures) can be dynamically imaged with a technique called angiography.

In fluoroscopic x-ray angiography, the patient is positioned between an x-ray source and a detector, a chemical contrast agent is injected into the vascular system of the patient as a bolus, and a sequence of x-ray images are captured as the chemical contrast agent travels through the vasculature. The chemical contrast agent may include any one or more of a plurality of chemical substances in liquid form. The chemical contrast agent is denser than blood or tissue; therefore, the chemical contrast agent attenuates the passage of x-rays more than blood or tissue. The denser the agent, the sharper the image contrast imparted on the containing vessel during fluoroscopic angiographic imaging. Some particularly dense formulations of chemical contrast agent contain iodine in its ionic form; this class of agents is termed “iodinated contrast.” One example of iodinated contrast is iohexol. Other chemical contrast agents do not have iodine and are not iodinated.

The chemical contrast agent traveling through the vasculature blocks the passage of the x-rays thereby creating an impression of the contrast-containing vascular structures on the detector. The resulting spatiotemporal x-ray attenuation pattern creates a sequence of x-ray images fluoroscopically obtained on the x-ray detector at a given frame rate. The sequence may be referred to as an angiogram, e.g., a sequence of images that trace the passage of the bolus of contrast. Angiograms are typically two-dimensional in space and one-dimensional in time.

The injected chemical contrast agent has toxic side effects to kidneys and other internal organs. However, lowering the dose of contrast agent to reduce the risk of these toxic side effects may produce unsatisfactory images with poor signal to noise ratios which, in turn, may lead to incomplete angiographic studies with inadequately imaged vascular anatomy. Use of a lower dose of contrast agent may also compel the advancement of the injecting catheter further into the arterial tree so that the injected contrast remains concentrated within the anatomic region of interest. The need to advance the injecting catheter further elevates the risk of complications caused by the catheter injuring ever smaller vessels distal in the vascular tree.

### SUMMARY

Aspects of the invention are directed to methods, systems, and computer readable media for angiographic dose reduction using machine learning (e.g., deep learning) with a concordance metric. A first aspect relates to a method for training a machine learning system to facilitate real time adjustment of a chemical contrast agent dosage or an x-ray radiation dosage during angiographic imaging using machine learning models with a concordance metric. In an embodiment, the method comprises providing, as an input to a first machine learning model, via a processor: a first set of one or more angiographic images obtained using a first chemical contrast agent dosage and a first x-ray radiation dosage; and a concordance metric indicating a degree of similarity between an estimated segmentation of the first set of one or more angiographic images generated by a second machine learning model and one or more segmented benchmark angiographic images provided as inputs to the second machine learning model, wherein the segmented benchmark angiographic images were obtained using a second chemical contrast agent dosage or a second x-ray radiation dosage that is higher than the first chemical contrast agent dosage or the first x-ray radiation dosage, respectively. The method may include generating, as an output of the first machine learning model, via a processor, an estimated concordance metric for the first set of one or more angiographic image. The method may include comparing, via a processor, the estimated concordance metric and the inputted concordance metric. The method may include performing, via a processor, a back propagating step to adjust parameters of the first machine learning model based on the comparing step.

In an embodiment, the concordance metric may be a loss metric. In an embodiment, the concordance metric may further include a loss metric slope. In an embodiment, the concordance metric may be a Dice coefficient. In an embodiment, the concordance metric may further include a Dice coefficient slope.

In an embodiment, the providing step may further include providing, as an input to the first machine learning model, via a processor, a dosage value based on the first chemical contrast agent dosage or the first x-ray radiation dosage used to obtain the first set of one or more angiographic images.

In an embodiment, the first and second machine learning models may be separate machine learning models.

A second aspect relates to a method of acquiring angiographic images with real time dose adjustment. An embodiment of the method comprises obtaining, via an angiographic imaging device, a third set of one or more angiographic images using a third chemical contrast agent dosage and a third x-ray radiation dosage. The method may include providing, via a processor, the third set of one or more angiographic images to a first machine learning model trained according to a method for training a machine learning system to facilitate real time adjustment of a chemical contrast agent dosage or an x-ray radiation dosage during angiographic imaging disclosed herein. The method may include generating, with the first machine learning model, via the processor, an estimated concordance metric based on the third set of one or more angiographic images. In an embodiment, the method may further include generating, via a processor, a suggested adjustment to the third chemical contrast dosage or the third x-ray radiation dosage based on the generated concordance metric. In an embodiment, the method may further include obtaining, via the angiographic imaging device, a fourth set of one or more angiographic images using a fourth chemical contrast dosage and a fourth x-ray radiation dosage based on the generated concordance metric. In an embodiment, the method may further include providing, as an input to a second machine learning model, via a processor, the fourth set of one or more angiographic images and generating, with the second machine learning model, via a processor, an estimated segmentation of the fourth set of one or more angiographic images. In an embodiment, the method may further include adjusting, via a processor, an x-ray radiation dosage emitted by the angiographic imaging device based on the generated concordance metric. In an embodiment, the method may further include adjusting, via a processor, a chemical contrast agent dosage administered by an autoinjector based on the generated concordance metric.

A third aspect relates to a system for real time adjustment of a chemical contrast agent dosage or an x-ray radiation dosage during angiographic imaging. An embodiment of the system comprises one or more processors, and a memory storing instructions executable by the one or more processors to obtain one or more angiographic images acquired using a first chemical contrast agent dosage and a first x-ray radiation dosage; provide the one or more angiographic images to a first machine learning model trained to generate a concordance metric indicative of a quality of an estimated segmentation of the one or more angiographic images by a second machine learning model trained to segment angiographic images; and cause the chemical contrast dosage or the x-ray radiation dosage to be adjusted based on the generated concordance metric.

A fourth aspect relates to a computer program product, an embodiment of which comprises one or more non-transitory computer readable storage media encoded with instructions that, when executed by one or more processors, cause the one or more processors to obtain one or more angiographic images acquired using a first chemical contrast agent dosage and a first x-ray radiation dosage; provide the one or more angiographic images to a first machine learning model trained to generate a concordance metric indicative of a quality of an estimated segmentation of the one or more angiographic images by a second machine learning model trained to segment angiographic images; and cause the chemical contrast dosage or the x-ray radiation dosage to be adjusted based on the generated concordance metric.

In an embodiment, the concordance metric may be a Dice coefficient. In an embodiment, the concordance metric may further include a Dice coefficient slope.

In an embodiment, the concordance metric may be compared with a predetermined value, such as a minimum acceptable probability of concordance. In an embodiment, the chemical contrast agent dosage or the x-ray radiation dosage may be decreased if the concordance metric is greater than the minimum acceptable probability of concordance.

In an embodiment, the concordance metric may be determined for an individual angiographic image frame and used to adjust dosage for a subsequent angiographic image frame. In another embodiment, the concordance metric may be determined for a plurality of angiographic image frames and used to adjust dosage for a subsequent plurality of angiographic image frames.

In an embodiment, a chemical contrast agent dosage administered by an autoinjector may be adjusted based on the generated concordance metric. In an embodiment, a x-ray dosage administered by an angiographic imaging device may be adjusted based on the generated concordance metric. In an embodiment, a suggested dosage adjustment may be communicated to a health care provider.

In yet another aspect, a method may be provided wherein angiographic image quality of comparatively earlier angiographic image frames is assessed during an angiographic study in order to adjust the radiation dose for subsequent angiographic image frames. In such a method, the chemical contrast may be injected into the vascular system at the start of an angiographic imaging sequence and may not be subject to real-time adjustment over the course of the angiographic image acquisition. However, the radiation dose may be adjusted with fine temporal resolution, e.g., between the acquisition of one image frame and the next during the angiogram. The image quality in angiographic image frames may be exploited to adjust the radiation dose in subsequent frames. Adjustment of angiographic x-ray dose within the course of an angiographic sequence may be referred to as “real-time” adjustment of x-ray dose.

Other objects and advantages of the techniques disclosed herein will be apparent from the specification and drawings.

### BRIEF DESCRIPTION OF THE DRAWINGS

FIGS. 1A and 1B are side and partially schematic views, respectively, showing an example of a rotational x-ray system that may be used with embodiments of the disclosure for acquiring angiographic data.

FIG. 2 is a schematic diagram of a computer system or information processing device that may be used with embodiments of the disclosure.

FIGS. 3A-3C each illustrate a flowchart of a method for training a neural network based on respective training data, according to an example embodiment.

FIG. 4 illustrates a system configured to generate simulated images of vascular structures acquired at lower doses of chemical contrast and/or x-ray radiation, according to an example embodiment.

FIG. 5 illustrates an example method for segmenting vasculature objects in a single image from an angiogram acquired at low chemical contrast and/or x-ray doses, according to an example embodiment.

FIG. 6 illustrates generating a higher-quality angiographic image from a target angiographic image that is located in various positions within a sub-sequence of angiographic images, using machine learning according to an example embodiment.

FIG. 7 illustrates example Dice coefficient data variability during training of a machine learning model with angiographic images.

FIG. 8 illustrates a schematic comparison of example image segmentations obtained with different x-ray doses at different Dice coefficients and Dice coefficient slopes.

FIG. 9 illustrates a graph of Dice coefficient as a function of x-ray dose.

FIG. 10 illustrates a schematic representation of a disclosed method for training a machine learning system on images of cardiac blood vessels.

FIG. 11 illustrates a schematic machine learning model for use in an angiographic evaluation.

FIG. 12 is a flowchart illustrating a method of training a machine learning system to facilitate real time adjustment of a chemical contrast agent dosage or an x-ray radiation dosage during angiographic imaging according to an example embodiment.

FIG. 13 is a flowchart illustrating a method of acquiring angiographic images with real time dose adjustment according to an example embodiment.

FIG. 14 shows actual angiographic images obtained with and without image processing according to an example embodiment.

FIG. 15 is a schematic diagram of a graphical user interface and controls in the form of foot pedals for controlling an amount of x-ray radiation and an imaging frame rate of an x-ray imaging device according to an example embodiment.

### DETAILED DESCRIPTION OF EXAMPLE EMBODIMENTS

Fluoroscopic angiographic imaging with chemical contrast is a commonly employed medical imaging procedure. Briefly, a physician secures access percutaneously into a blood vessel (commonly the femoral artery), navigates a catheter to the root artery of the organ in question, and injects the chemical contrast in temporal coordination with the fluoroscopic acquisition of a sequence of x-ray images.

Fluoroscopic angiography may be used to diagnose blocked coronary arteries of the heart or in the brain. The same angiographic study may offer an avenue for therapy if that study establishes the diagnosis of a blocked artery. For example, a stent may be placed across a calcified plaque in a coronary artery of the heart, or a thrombolytic medication may be applied directly to a blood clot in a cerebral artery.

An angiographic imaging study or angiogram may include target objects in the foreground, such as blood vessels. The greater the sharpness, detail, and clarity with which foreground objects in the imaging study are displayed relative to the background, the greater the signal to noise ratio of the imaging study and, therefore, the greater the diagnostic value. But in some cases, fluoroscopic angiography can provide insufficient clarity to the configuration of blood vessels in an angiographic image, even when chemical contrast is injected to improve definition of the blood vessels in the anatomic area being fluoroscopically imaged.

In standard practice, the image quality of the angiographic study can be improved by increasing the dose of the injected chemical contrast and/or by increasing the dose of the fluoroscopic x-ray radiation. However, use of chemical contrast and x-ray radiation in an angiographic imaging study can carry risk. For example, there is risk associated with the procedure of placing a catheter into a blood vessel and navigating it to the organ of interest for chemical contrast injection. In addition, the chemical contrast agent and the x-ray radiation can be harmful to (e.g., cause toxic side-effects in) a human or animal study subject.

All chemical contrast agents can have a measure of toxicity when injected into the vascular circulation system. Upon injection, the chemical contrast agent may begin to be cleared by biochemical and physiological processes, led by clearing organs such as the kidneys and the liver. The chemical contract agent may be toxic to the clearing organs in a dose-dependent manner. The chemical contrast agent can also produce a significant mass load into the vascular system, producing stress on the heart and other vascular structures, potentially inducing or aggravating heart failure in a patient with compromised heart pump action. The agent can also place stress on the clearing organs.

Also, some humans may develop immune reactions to specific molecular structures on various chemical contrast agents, particularly on iodinated contrast agents. A histiocyte-mediated immune reaction to an injected contrast agent may be immediate and severe, and can be fatal if not promptly recognized and treated. For at least these reasons, increasing the chemical contrast to obtain a higher-quality angiography study also increases the risk of harm to the subject.

In addition, x-ray doses can be harmful to irradiated tissues, particularly in tissues that are particularly sensitive to radiation, such as the thyroid gland and reproductive organs. Radiation doses may induce chronic inflammation and/or injure constituent bio-molecules of tissues, potentially leading to consequences ranging from comparatively minor skin irritation along the x-ray path to cancer formation at irradiated tissues. Higher x-ray doses in particular can directly injure the bio-molecular constituents of tissue, including deoxyribonucleic acid (DNA) in the cell nuclear apparatus, producing malformations of organs and carrying a risk of neoplasia.

For a given angiogram, there is an x-ray dose that optimizes the image quality. A lower x-ray does not penetrate the anatomy in sufficient quantity to form a quality image on the x-ray sensor. In an extremely low x-ray dose, all of the pixels are black, none having received any x-rays. A higher dose saturates the x-ray sensor. In an extremely high x-ray dose, all of the pixels are bleach white. In the context of fluoroscopic angiography, to segment an image means to increase the intensity of the particular pixels that represent blood vessels. Since the blood vessels are the objects of interest in an angiogram, enhanced segmentation is a form of enhanced image quality. There are machine learning methods based on deep neural networks for enhancing angiographic image quality by means of segmentation.

Similarly, there is an injected chemical contrast dose that optimizes image quality. A lower contrast dose may not produce distinct blood vessel patterns in the image if the blood vessel contents do not block passing x-rays more than surrounding tissue. A higher dose may hide patterns within blood vessels. A higher contrast dose may be matched with a higher x-ray dose to increase the distinction in the angiographic images of blood vessels.

Both x-ray and chemical contrast doses have dose-dependent toxicity. Higher x-ray doses can induce inflammation in irradiated tissues in the short term. Higher X-ray doses can produce serious later-term side effects including cancer. Chemical contrast is toxic to the kidneys and may be tolerated poorly in patients with renal insufficiency. Even small chemical contrast doses may be harmful or fatal to patients with chemical contrast hypersensitivity or allergy.

Accordingly, techniques are described herein to reduce the dosage of chemical contrast and/or x-ray radiation that are administered during an angiographic procedure. In particular, angiographic image quality may be conserved by displaying the imaged vasculature at greater sharpness and accuracy in the foreground using lower/reduced doses both of chemical contrast and x-ray radiation. As described in greater detail below, this may be accomplished using machine learning (e.g., deep learning) techniques with concordance metrics, such as loss metrics associated with the image quality.

Embodiments of the invention are directed to methods, systems, and computer readable media for angiographic dose reduction using machine learning (e.g., deep learning). Briefly, techniques described herein may use deep learning to conserve/preserve angiographic image quality while reducing the angiographic dose of potentially harmful x-ray radiation. Furthermore, techniques are described to conserve angiographic image quality in real-time, allowing the reduction of angiographic x-ray dose within a same angiographic fluoroscopic sequence. As a result, angiographic anatomy may be extracted from an image at reduced angiographic doses.

Various mechanisms are proposed herein to train a machine learning model (e.g., a deep learning neural network) to produce higher-quality or segmented images with reduced angiographic dosage. These mechanisms may involve gathering reduced dose training data crossed with standard dose segmented data. That is, a deep neural network training data set may be generated using full-dose chemical contrast and x-ray radiation to compose segmented data and reduced dose data to train the neural network against the full-dose segmented data. The reduced dose training data may be generated using: full (standard) and physically reduced doses in animal (e.g., non-human) angiograms; full and physically reduced doses in realistic artificial models of organs; and/or by computationally simulated reduction of doses in full dose human angiographic data. Thus, in a deep learning neural network training step, a plurality of angiographic images acquired across a temporal interval may be employed to segment blood vessels. A deep learning neural network (e.g., a convolutional network) may obtain the standard dose segmentation results with reduced dose image data. The deep learning neural network may be trained against training data with high-quality angiographic structure ground-truth data and actual or simulated degraded angiographic images captured with reduced chemical contrast or x-ray radiation doses. There may be a minimal quality of the segmented angiographic images that by clinical judgment and experience satisfies the requirement for the diagnostic and therapeutic purposes of the angiographic study. A real-time model for estimating the minimal x-ray dose needed to satisfy the desired image quality is applied in real-time over the course of the angiographic study.

The deep learning neural network may be applied to a span or sequence of angiographic images to identify vascular structure in an angiographic image frame in relation to the given angiographic dose. The deep learning neural network may estimate the vascular structures in a given image from the target image and from one or more temporally preceding images. The deep network architecture may be tuned to detect spatiotemporal properties of contrast-containing blood vessels.

There is a relationship between angiographic X-ray dose and the quality of segmented angiographic images that, in an embodiment, may be calculated from the above machine learning model. In an embodiment, this relationship may be subject to a further machine learning model, such that for a future angiographic image, the quality of the segmented result, and the change of the quality of the segmented result may be predicted from a change in radiation dosage.

In an embodiment, a neural network machine learning model for producing segmented angiographic images is provided, and there is an angiographic image frame or sequence of temporally adjacent angiographic sequence frames that are supplied as the input to the neural network. In an embodiment, there are layers of neurons encoded in software that are connected by floating point numbers. There is an output of a segmented image frame. The tasks associated with the neural network may be divided into a training phase and an execution phase. During a training phase, the output of the segmented image frame may be compared to a gold standard (usually human hand-coded) segmented angiographic image. The difference between these two may be used in a back propagation step to adjust the floating point connections between the neurons in the layers of the deep network. In an embodiment, there is a summary floating point number that describes the closeness of correspondence of the computed segmented image to the gold-standard segmented image. In an example embodiment, this number is a Dice coefficient. See Dice, L. R., “Measures of the amount of ecologic association between species,” Ecology, 26 (3): 297-302 (1945).

During an execution phase, an angiographic image or sequence of temporally adjacent angiographic images is supplied as the input to the trained neural network. Based on computation from the floating point numbers that represent the connections between the neurons as generated in the training phase, a segmented image is inferred. In one embodiment, in addition to training a neural network to estimate the segmented image, there is a neural network that is trained to estimate the Dice coefficient. Conceptually, these are two different neural networks that may operate in parallel. There are embodiments where the estimation of the segmented image and of the Dice coefficient may be performed by a single expanded neural network.

An angiogram is typically acquired at between 4 and 30 images per second. In an embodiment, the neural network that produces the Dice coefficient is preferably tuned in its complexity and by the availability of floating point hardware to produce the Dice coefficient in apparent real time, which is to produce it at a rate that approximates that of the angiographic sampling. In an embodiment, the estimated Dice coefficient produced by the neural network in real-time may be compared to the previously characterized relationship between x-ray dose and Dice coefficient. It may furthermore be compared to the previously characterized coefficient between Dice coefficient and the necessary image quality to satisfy the diagnostic and treatment objectives of the angiographic session. The Dice coefficient may be applied through these previously characterized relationships to automatically adjust the radiation dose to give the minimal dose consistent with the satisfaction of the clinical objectives.

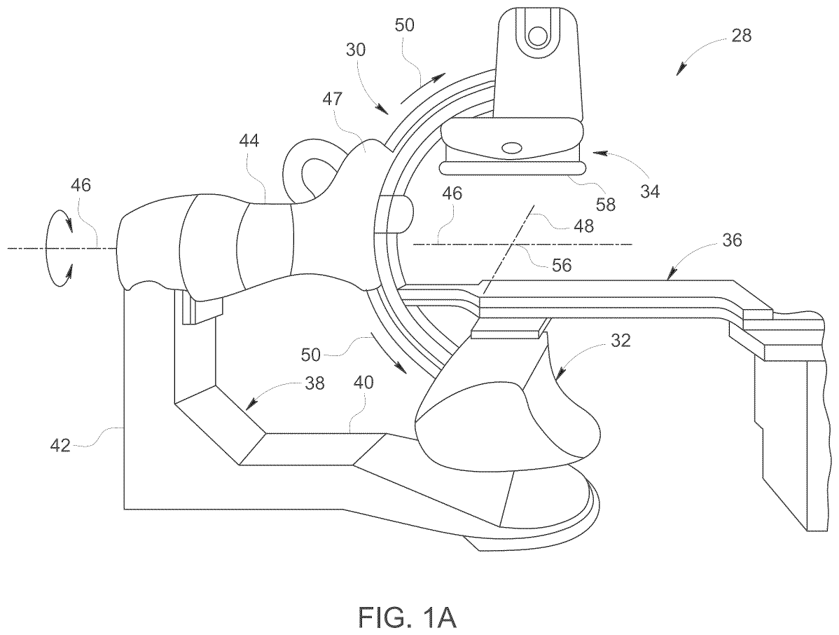

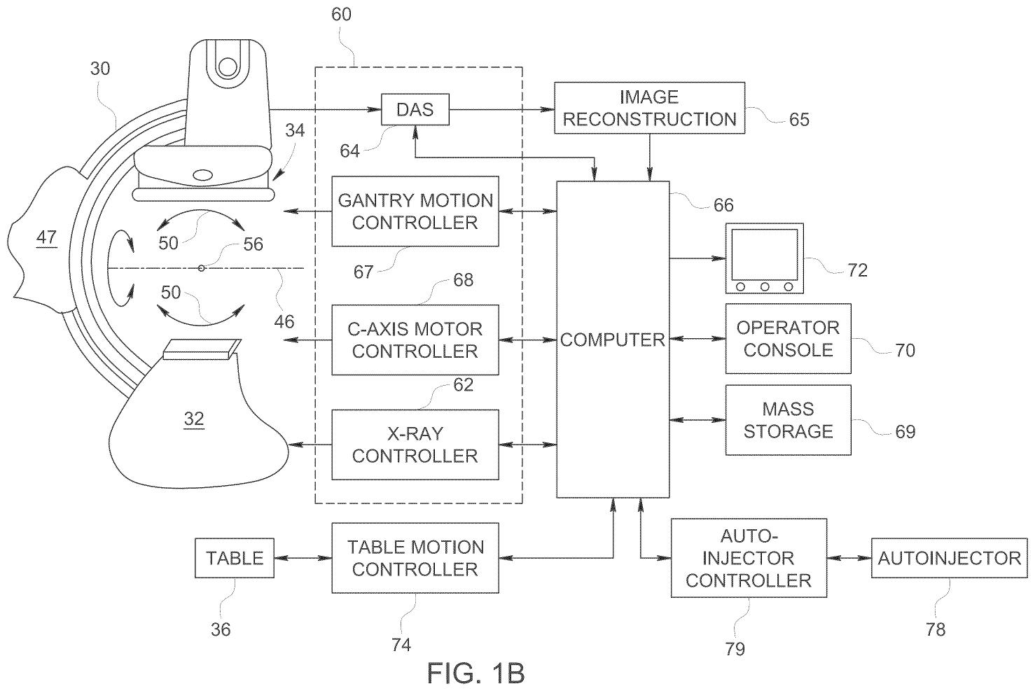

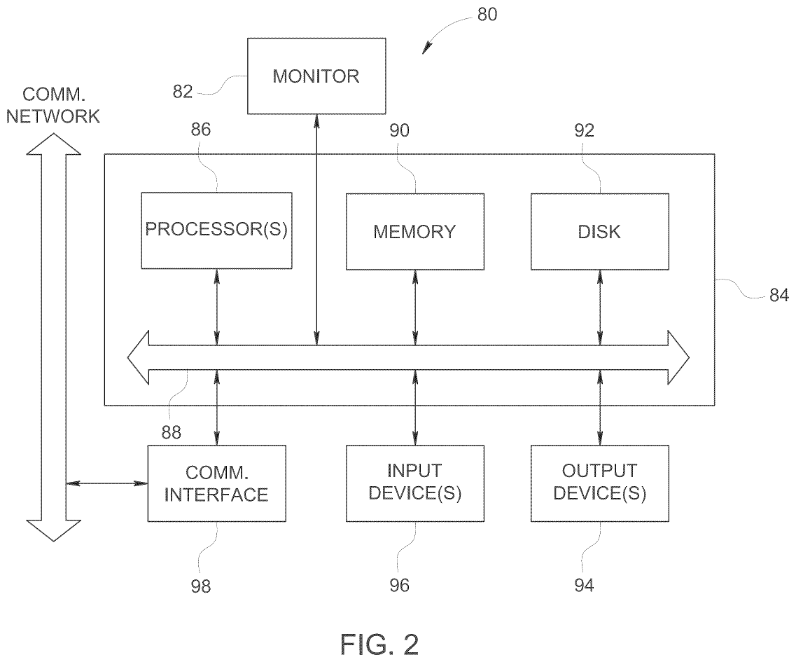

Referring to FIGS. 1A, 1B, and 2, exemplary systems or devices that may be employed for carrying out embodiments of the invention are illustrated. It is understood that such systems and devices are only exemplary of representative systems and devices and that other hardware and software configurations are suitable for use with embodiments of the invention. Thus, the embodiments are not intended to be limited to the specific systems and devices illustrated herein, and it is recognized that other suitable systems and devices can be employed without departing from the spirit and scope of the subject matter provided herein.

Referring first to FIGS. 1A and 1B, a rotational x-ray system 28 is illustrated that may be employed for obtaining an angiogram via fluoroscopic angiography. In acquiring an angiogram, a chemical contrast agent may be injected into the patient positioned between an x-ray source and detector, and x-ray projections are captured by the x-ray detector as a two-dimensional image (i.e., an angiographic image or image frame). A sequence of such image frames comprises an angiographic study. In one example, the sequence of angiographic image frames may be obtained at a rate faster than the subject's cardiac rate. For example, the subject's cardiac rate may be measured (e.g., using an EKG device or the like) and the sequence of angiographic image frames may be obtained at a rate faster than the measured cardiac rate.

As shown in FIG. 1A, an example of an angiographic imaging system is shown in the form of a rotational x-ray system 28 including a gantry having a C-arm 30 which carries an x-ray source assembly 32 on one of its ends and an x-ray detector array assembly 34 at its other end. The gantry enables the x-ray source assembly 32 and x-ray detector array assembly 34 to be oriented in different positions and angles around a patient disposed on a table 36, while providing to a physician access to the patient. The gantry includes a pedestal 38 which has a horizontal leg 40 that extends beneath the table 36 and a vertical leg 42 that extends upward at the end of the horizontal leg 40 that is spaced apart from table 36. A support arm 44 is rotatably fastened to the upper end of vertical leg 42 for rotation about a horizontal pivot axis 46.

The horizontal pivot axis 46 is aligned with the centerline of the table 36, and the support arm 44 extends radially outward from the horizontal pivot axis 46 to support a C-arm drive assembly 47 on its outer end. The C-arm 30 is slidably fastened to the C-arm drive assembly 47 and is coupled to a drive motor (not shown) which slides the C-arm 30 to revolve about a C-axis 48 as indicated by arrows 50. The horizontal pivot axis 46 and C-axis 48 intersect each other, at a system isocenter 56 located above the table 36, and are perpendicular to each other.

The x-ray source assembly 32 is mounted to one end of the C-arm 30 and the x-ray detector array assembly 34 is mounted to its other end. The x-ray source assembly 32 emits a beam of x-rays which are directed at the x-ray detector array assembly 34. Both assemblies 32 and 34 extend radially inward to the horizontal pivot axis 46 such that the center ray of this beam passes through the system isocenter 56. The center ray of the beam thus can be rotated about the system isocenter around either the horizontal pivot axis 46 or the C-axis 48, or both, during the acquisition of x-ray attenuation data from a subject placed on the table 36.

The x-ray source assembly 32 contains an x-ray source which emits a beam of x-rays when energized. The center ray passes through the system isocenter 56 and impinges on a two-dimensional flat panel digital detector 58 housed in the x-ray detector array assembly 34. The two-dimensional flat panel digital detector 58 may be, for example, a 2048×2048 element two-dimensional array of detector elements. Each element produces an electrical signal that represents the intensity of an impinging x-ray and hence the attenuation of the x-ray as it passes through the patient. During a scan, the x-ray source assembly 32 and x-ray detector array assembly 34 are rotated about the system isocenter 56 to acquire x-ray attenuation projection data from different angles. In some embodiments, the detector array is able to acquire fifty projections, or image frames, per second which is the limiting factor that determines how many image frames can be acquired for a prescribed scan path and speed.

Referring to FIG. 1B, the rotation of the assemblies 32 and 34 and the operation of the x-ray source are governed by a control mechanism 60 of the x-ray system. The control mechanism 60 includes an x-ray controller 62 that provides power and timing signals to the x-ray source assembly 32. A data acquisition system (DAS) 64 in the control mechanism 60 samples data from detector elements and passes the data to image reconstruction system or module 65. The image reconstruction system 65 receives digitized x-ray data from the DAS 64 and performs high speed image reconstruction according to the methods of the present disclosure. The reconstructed image is applied as an input to a computer 66 which stores the image in a mass storage device 69 or processes the image further. Image reconstruction system 65 may be included in a standalone computer or may be integrated with computer 66.

The control mechanism 60 also includes a gantry motor controller 67 and a C-axis motor controller 68. In response to motion commands from the computer 66, the motor controllers 67 and 68 provide power to motors in the x-ray system that produce the rotations about horizontal pivot axis 46 and the C-axis, respectively. The computer 66 also receives commands and scanning parameters from an operator via console 70 that has a keyboard and other manually operable controls. An associated display 72 allows the operator to observe the reconstructed image frames and other data from the computer 66. The operator supplied commands are used by the computer 66 under the direction of stored programs to provide control signals and information to the DAS 64, the x-ray controller 62 and the motor controllers 67 and 68. In addition, computer 66 operates a table motor controller 74 which controls the motorized table 36 to position the patient with respect to the system isocenter 56.

FIG. 1B also shows an optional autoinjector 78 for administering a chemical contrast agent to a patient during an angiographic study. The autoinjector 78 may include a reservoir for holding a chemical contrast agent and a pump for conveying chemical contrast agent from the reservoir to the patient via an intravenous (“IV”) line or catheter. An optional autoinjector controller 79, in communication with the computer 66, may provide control signals to the autoinjector 78 to control the amount of chemical contrast agent administered to the patient. In an example embodiment, the computer 66 is configured to communicate with the autoinjector 78 via the autoinjector controller 79 to adjust the amount of chemical contrast agent being administered in real-time (e.g., during an angiographic procedure) based on output from a machine learning model trained according to embodiments of the present invention. For example, the amount of chemical contrast agent administered to the patient by the autoinjector 78 may be adjusted by sending control signals to the autoinjector controller 79 that cause the autoinjector to increase or decrease a flow rate of the chemical contrast agent.

Referring now to FIG. 2, a block diagram of a computer system or information processing device 80 (e.g., image reconstruction system 65 and/or computer 66 in FIG. 1B) is illustrated that may be incorporated into an angiographic imaging system, such as the rotational x-ray system 28 of FIGS. 1A and 1B, and/or may be used as a standalone device, for angiographic dose adjustment using machine learning according to embodiments of the present invention. Information processing device 80 may be local to or remote from rotational x-ray system 28. In one example, the functionality performed by information processing device 80 may be offered as a Software-as-a-Service (SaaS) option. SaaS refers to a software application that is stored in one or more remote servers (e.g., in the cloud) and provides one or more services (e.g., angiographic image processing) to remote users. Angiographic images may be obtained directly from an angiographic imaging system, such as the rotational x-ray system 28 of FIGS. 1A and 1B, or from other sources such as physical storage media configured to store data.

In one embodiment, computer system 80 includes a monitor or display device 82, a computer system 84 (which includes processor(s) 86, bus subsystem 88, memory subsystem 90, and disk subsystem 92), user output devices 94, user input devices 96, and communications interface 98. Monitor 82 can include hardware and/or software elements configured to generate visual representations or displays of information. Some examples of monitor 82 may include familiar display devices, such as a television monitor, a cathode ray tube (CRT), a liquid crystal display (LCD), a light-emitting diode (LED) display, or the like. In some embodiments, monitor 82 may provide an input interface, such as incorporating touch screen technologies.

Computer system 84 can include familiar computer components, such as one or more central processing units (CPUs), memory or storage devices, graphics processing units (GPUs), communication systems, interface cards, or the like. As shown in FIG. 2, computer system 84 may include at least one hardware processor 86 that communicates with a number of peripheral devices via bus subsystem 88. Processor(s) 86 may include commercially available central processing units or the like. Bus subsystem 88 can include mechanisms for letting the various components and subsystems of computer system 84 communicate with each other as intended. Although bus subsystem 88 is shown schematically as a single bus, alternative embodiments of the bus subsystem may utilize multiple bus subsystems. Peripheral devices that communicate with processor(s) 86 may include memory subsystem 90, disk subsystem 92, user output devices 94, user input devices 96, communications interface 98, or the like.

Processor(s) 86 may be implemented using one or more analog and/or digital electrical or electronic components, and may include a microprocessor, a microcontroller, an application-specific integrated circuit (ASIC), a field programmable gate array (FPGA), programmable logic and/or other analog and/or digital circuit elements configured to perform various functions described herein, such as by executing instructions stored in memory subsystem 90 and/or disk subsystem 92 or another computer program product.

Memory subsystem 90 and disk subsystem 92 are examples of physical storage media configured to store data, such as instructions executable by the one or more processors 86 to perform the operations described herein. Memory subsystem 90 may include a number of memories or memory devices including random access memory (RAM) for volatile storage of program code, instructions, and data during program execution and read only memory (ROM) in which fixed program code, instructions, and data are stored. Disk subsystem 92 may include a number of file storage systems providing persistent (non-volatile) storage for programs and data. Other types of physical storage media include floppy disks, removable hard disks, optical storage media such as compact disc-read-only memories (CD-ROMS), digital video disc (DVDs) and bar codes, semiconductor memories such as flash memories, read-only-memories (ROMS), battery-backed volatile memories, networked storage devices, or the like. Memory subsystem 90 and disk subsystem 92 may be configured to store programming and data constructs that provide functionality or features of techniques discussed herein. Software code modules and/or processor instructions that when executed by processor(s) 86 implement or otherwise provide the functionality may be stored in memory subsystem 90 and disk subsystem 92. Memory subsystem 90 and/or disk subsystem 92 may be a non-transitory computer readable storage medium.

User input devices 96 can include hardware and/or software elements configured to receive input from a user for processing by components of computer system 80. User input devices can include all possible types of devices and mechanisms for inputting information to computer system 84. These may include a keyboard, a keypad, a touch screen, a touch interface incorporated into a display, audio input devices such as microphones and voice recognition systems, and/or other types of input devices. In various embodiments, user input devices 96 may include a computer mouse, a trackball, a track pad, a joystick, a wireless remote, a drawing tablet, a voice command system, an eye tracking system, or the like. In some embodiments, user input devices 96 are configured to allow a user to select or otherwise interact with objects, icons, text, or the like that may appear on monitor 82 via a command, motions, or gestures, such as a click of a button or the like.

User output devices 94 can include hardware and/or software elements configured to output information to a user from components of computer system 80. User output devices can include all possible types of devices and mechanisms for outputting information from computer system 84. These may include a display device (e.g., monitor 82), a printer, a touch or force-feedback device, audio output devices, or the like.

Communications interface 98 can include hardware and/or software elements configured to provide unidirectional or bidirectional communication with other devices. For example, communications interface 98 may provide an interface between computer system 84 and other communication networks and devices, such as via an internet connection.

Techniques described herein may enable reduction of the angiographic doses (e.g., chemical contrast agent doses and/or x-ray radiation doses) required to obtain a diagnostically useful angiographic image. In particular, the angiographic doses may be reduced compared to standard/conventional angiographic doses that would otherwise be required to obtain a diagnostically useful angiographic image in the absence of the techniques described herein (e.g., in the absence of a machine learning model, such as a deep learning neural network, as described herein). In one example, a “diagnostically useful” or “high-quality” angiographic image provides data of a quality sufficient to provide meaningful clinical information and/or to allow treatment decisions to be made, e.g., a diagnostically useful angiographic image may be one of sufficient clarity to allow health care professionals to visually identify and segment vessels in the image. For example, if a person has chest pain due to insufficient blood flow to the coronary arteries that supply blood to the heart muscle, a diagnostically useful angiographic image of the coronary arteries may accurately display the segment of the coronary artery of the heart with a stenosis that impairs circulation to the heart muscle.

The x-ray dosage required to generate a diagnostically useful image in an angiogram also varies depending on physical characteristics of the patient/subject and the nature of the angiographic procedure. Methods of calculating x-ray dosages are well known in the art. Typically, a full or standard dose of x-ray radiation for an interventional cardiac procedure in a fluoroscopic unit ranges from a minimum of 8 milliSieverts of radiation to 10 milliSieverts of radiation. A Sievert is the equivalent of 1 Joule of energy per kilogram of mass. The ionizing nature of x-ray radiation means that it is always desirable to minimize the exposure of the subject (and medical staff associated with an angiography procedure) to x-rays as much as possible while still producing a useful visualization of the target tissue.

Chemical contrast agents are chemical compounds containing x-ray attenuating atoms. The main type of chemical contrast agent used in angiography is the family of iodinated contrast agents, which can be ionic or, advantageously, non-ionic iodinated contrast agents. Such contrast agents are well known in the art and include: iohexol (Omnipaque™, GE Healthcare); iopromide (Ultravist™, Bayer Healthcare); iodixanol (Visipaque™, GE Healthcare); ioxaglate (Hexabrix™, Mallinckrodt Imaging); iothalamate (Cysto-Conray II™, Mallinckrodt Imaging); and iopamidol (Isovue™, Bracco Imaging). See also Lusic and Grinstaff, “X-Ray Computed Tomography Contrast Agents,” Chem Rev. 13:1641-66 (2013). Examples of other agents include gadolinium-based agents. See Ose et al., “‘Gadolinium’ as an Alternative to Iodinated Contrast Media for X-Ray Angiography in Patients With Severe Allergy,” Circ J. 2005; 69:507-509 (2005).

The standard or full-dosages for such chemical contrast agents vary depending on the nature of the agent, the physical characteristics of the patient/subject, and the nature of the angiographic procedure. In general, however, the standard or full-dosages of such chemical contrast agents are the minimum dosage required to improve the visualization of the target tissue by increasing the difference in absolute CT (computerized tomography) attenuation value between the target tissue and surrounding tissue and fluids by a certain amount. For fluoroscopic angiography, a full dosage of the injected chemical contrast agent typically increases the CT attenuation value of a blood vessel somewhere between 2× and 10× the baseline level without the chemical contrast agent. In fluoroscopic angiography, the injection catheter is navigated near to the target organ, allowing a higher local dose but a lower total body dose. In CT, where the contrast is injected intravenously, the contrast is diluted throughout the vascular system. Thus, the local concentration of contrast in any given blood vessel may be lower. The increase in CT attenuation value from such an intravenous injection of a chemical contrast agent is typically between 1.2× and 4× the baseline CT attenuation value without the chemical contrast agent. An increase of at least 1.2× in CT attenuation from baseline for CT angiography, and at least 2× in CT attenuation from baseline for fluoroscopic angiography, have been found to provide “diagnostically useful” or “high-quality” angiographic images. The contrast agent should preferably contain a high mol % of the x-ray attenuating atom per agent (molecule, macromolecule, or particle) in order to reduce the volume used and concentrations needed for imaging. Also, the tissue retention-time of the contrast agent should preferably be sufficiently long for completion of a CT scan and scheduling the instrument time in the diagnostic setting (e.g., 2-4 h). Moreover, the contrast agent preferably should: (a) localize or target the tissue of interest and possess favorable biodistribution and pharmacokinetic profiles; (b) be readily soluble or form stable suspensions at aqueous physiological conditions (appropriate pH and osmolality) with low viscosity; (c) be non-toxic; and (d) be cleared from the body in a reasonably short amount of time, usually within several hours (<24 h).

In some embodiments, the angiographic iodinated contrast dose used to obtain diagnostically useful angiographic images may be reduced by about 25% of the standard or full-dosage amount of contrast that is typically injected. Further dose reduction may be achieved using the techniques described herein in combination with spatiotemporal reconstruction techniques as described in U.S. application Ser. No. 16/784,073, filed on Feb. 6, 2020, which is incorporated by reference herein in its entirety. The spatiotemporal reconstruction of an image may be an input to the machine learning model, or the output of the machine learning model may be processed using spatiotemporal reconstruction.

To increase the sharpness and clarity of the imaged vasculature at lower doses of chemical contrast and/or lower doses of x-ray radiation, a deep learning neural network (e.g., having an input layer, an output layer, and three or more layers between the input and output layers) is provided with properties that promote its performance in detecting vasculature at low chemical contrast and/or x-ray doses. While descriptions provided herein focus on deep learning neural networks, it will be appreciated that these techniques may be utilized with any suitable machine learning model, of which a deep learning neural network is just one example. The machine learning model may be implemented by any suitable machine learning techniques (e.g., mathematical/statistical, classifiers, feed-forward, recurrent, convolutional or other neural networks, etc.). For example, a neural network may be used that includes an input layer, one or more intermediate layers (e.g., including any hidden layers), and an output layer. Each layer may include one or more nodes or neurons, where the input layer neurons receive input (e.g., image data, feature vectors of images, etc.), and may be associated with weight values. For example, each node in the input layer may receive or be encoded with the relative brightness of one pixel of an angiographic image as an input, and the relative brightness may be a floating point number between 0 and 1. The neurons of the intermediate and output layers are connected to one or more neurons of a preceding layer, and receive as input the output of a connected neuron of the preceding layer. Each connection is associated with a weight value, and each neuron produces an output based on a weighted combination of the inputs to that neuron. The output of a neuron may further be based on a bias value for certain types of neural networks (e.g., recurrent types of neural networks). The weight (and bias) values may be adjusted based on various training techniques. For example, the machine learning of the neural network may be performed using a training set of reduced-dosage angiographic images as input and corresponding high-quality full-dosage angiographic images as known outputs, where the neural network attempts to produce the known output by using an error from the output produced by the neural network (e.g., difference between produced and known outputs) to adjust weight (and bias) values (e.g., via backpropagation or other training techniques). In example embodiments, the known output at each node may be a pixel value representing brightness or intensity (e.g., a floating point number between 0 and 1) or a probability (p-value) that the pixel is a vessel (e.g., a floating point number between 0 and 1). A neural network trained using the latter technique (i.e., wherein the known output is a segmented angiographic image with p-values of 0-1 for each pixel) can be advantageously used to produce a segmented angiographic image from reduced-dosage angiographic images.

More specifically, in a training phase, a data conditioning system may be provided to train the deep learning neural network to perform well in the setting of low chemical contrast and/or x-ray doses. Thus, the training phase may enable a deployment phase in which the deep learning neural network is able to output high-quality angiographic images based on angiographic images obtained at lower/reduced chemical contrast and/or lower/reduced x-ray radiation doses.

In one aspect, a deep learning network data training system is provided that promotes the ability of a convolutional spatiotemporal network to detect pixels corresponding to blood vessels despite the use of low doses of chemical contrast and/or fluoroscopic x-ray radiation. The deep learning neural network may be trained using angiographic training data. The angiographic training data may include (1) a first set of angiographic images obtained at conventional/standard chemical contrast and x-ray radiation doses, and (2) comparable angiographic images with reduced chemical contrast and/or x-ray radiation doses. In an embodiment, the comparable angiographic images with reduced chemical contrast and/or x-ray radiation doses may be used for the training set as inputs, and the angiographic images obtained at conventional/standard chemical contrast and x-ray radiation doses may be used as known outputs. In an embodiment, feature vectors may be extracted from the images and used with the corresponding known outputs for the training set as inputs. A feature vector may include any suitable features (e.g., pixel intensity, etc.).

In one example, the comparable angiographic image from the second set may be identical or nearly identical to a counterpart image in the first set, other than the difference in angiographic doses. For instance, the same object may be imaged in both angiographic images, and may have substantially the same size, position and orientation in both images. The angiographic image from the first set and the counterpart image from the second set may be similar enough to provide useful training data to the deep learning neural network, e.g., data that may help train the deep learning neural network to produce a diagnostically useful angiographic image based on an angiographic image that is obtained at reduced angiographic doses and not diagnostically useful.

The training data may be acquired based on any suitable angiographic training images. For example, the training data may be obtained (1) in a laboratory setting using animals undergoing approved angiographic studies; (2) in a laboratory setting using physical models with fluid mechanically pumped into synthetic organs which are angiographically imaged; and/or (3) from full-quality human clinical angiographic data that have been computationally modified to simulate lower chemical contrast and/or x-ray doses. The data training system may use these training options alone or in any suitable combination to train the deep learning neural network.

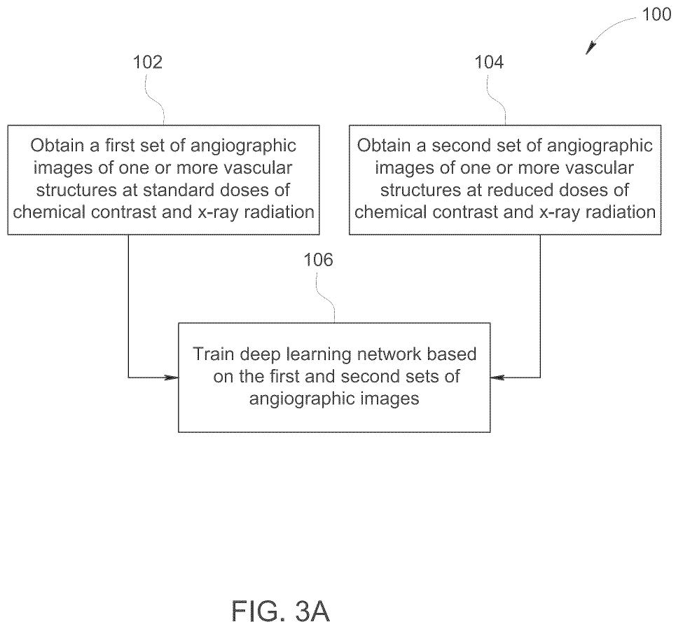

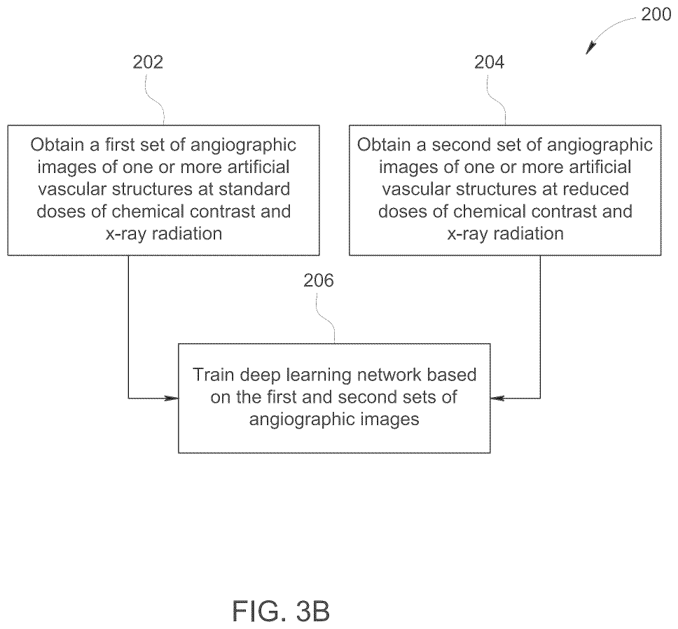

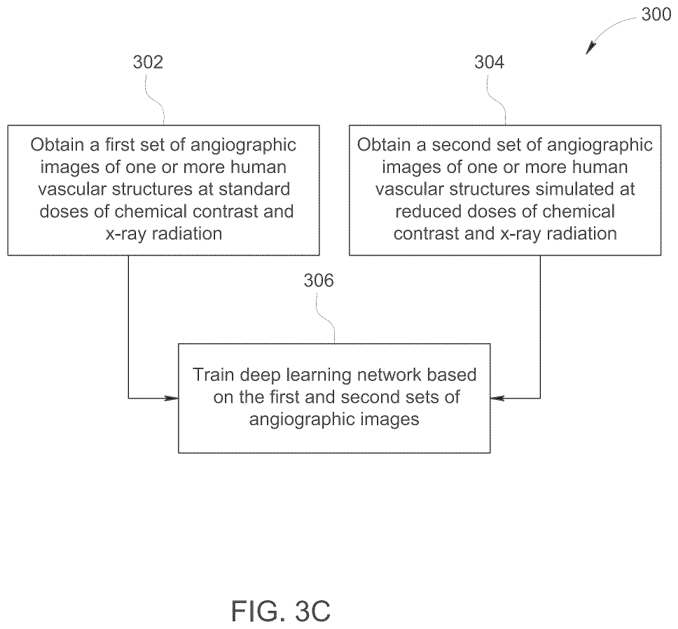

FIG. 3A illustrates a flowchart of an example method 100 for training a deep learning neural network based on vascular structures, such as non-human vascular structures (e.g., training data option (1)—training data obtained in a laboratory setting using animals undergoing approved angiographic studies). In this example, at step 102, a first set of angiographic images of one or more non-human vascular structures is obtained at standard/conventional doses of chemical contrast (e.g., for the coronary arteries, approximately 10 ml of 300 mg iodine/ml) and x-ray radiation (e.g., for an angiogram, a dose area product of approximately 400 dGy×cm²); at step 104, a second set of angiographic images of one or more non-human vascular structures is obtained at reduced doses of chemical contrast (e.g., ¼ of standard iodinated contrast doses) and/or x-ray radiation (e.g., ½ of standard radiation doses); and, at step 106, the deep learning neural network is trained based on the first and second sets.

In one example, laboratory animals may be used to obtain angiograms at conventional doses of chemical contrast and x-ray radiation in order to provide a “gold standard” or “benchmark” reference set of angiographic images. Then, without moving the animal or the gantry position of the fluoroscopic imaging unit, angiographic studies may be obtained at lower chemical contrast and/or x-ray doses.

The angiographic images obtained based on conventional doses may be manually segmented by human technicians to identify the blood vessels. These segmentations may serve as guides or “benchmarks” for the segmentations of blood vessels that are poorly seen in the stress angiograms that are similar or identical in every way except for being obtained at lower chemical contrast and x-ray doses. As used herein, the term “to segment” may refer to manually, semi-automatically, or fully-automatically identifying and representing (e.g., displaying) the vascular elements of an angiogram. A vascular “segmentation” may refer to processing of an image such that the vascular structures are represented as distinct from the noise and other structures in the imaged field of view. For example, the vascular structures in a “segmented” angiographic image may be represented as white objects against a black background. Angiographic images subject to segmentation may be referred to herein as “segmented angiographic images.” In an embodiment, segmented angiographic images obtained at conventional doses of chemical contrast and x-ray radiation may be used as training data (e.g., known outputs) with reference to the angiographic images obtained at lower doses.

FIG. 3B illustrates a flowchart of an example method 200 for training a deep learning neural network based on artificial vascular structures (e.g., training option (2)—training data obtained in a laboratory setting using physical models with fluid mechanically pumped into synthetic organs which are angiographically imaged). In this example, at step 202, a first set of angiographic images of one or more artificial vascular structures is obtained at standard doses of chemical contrast and x-ray radiation; at step 204, a second set of angiographic images of one or more artificial vascular structures is obtained at reduced doses of chemical contrast and x-ray radiation; and, at step 206, the deep learning neural network is trained based on the first and second sets.

The artificial vascular structures may be human-manufactured, solid vascular organ phantoms or organoids which include mechanical fluid pumps that simulate pulsatile arterial flow. The fluid may be pumped into the artificial vascular structure with a network of hollow tubular structures that share the size, shape, and branching pattern of the blood vessels. One manufacturer of artificial vascular structures is Heartroid, JMC Corporation, Yokohama, Japan. The artificial vascular structure may offer a suitable generator of systematic standard and low chemical contrast and x-ray dose images.

In one specific example, the organoid may be positioned in an angiographic imaging suite; fluid with chemical contrast may be injected; and fluoroscopic x-ray images may be obtained to generate a sequence of angiographic images of the chemical contrast flowing through the organoid vascular channels. Then, without changing the position of the organoid or of the imaging gantry, an interacting range of lower chemical contrast doses crossed with lower x-ray doses may be employed to obtain a large body of angiographic images at varying chemical contrast and x-ray doses. The angiographic images obtained at higher doses may serve as training data with reference to the angiographic images obtained at lower doses. Thus, the neural network may be offered the low chemical contrast and x-ray dose data for training with the vascular data obtained from the higher dose angiographic studies.

The images obtained at higher doses may be refined by human editing or by mathematical processing, e.g., using techniques discussed in A. F. Frangi, W. J. Niessen, K. L. Vincken, and M. A. Viergever, “Multiscale vessel enhancement filtering,” Lecture Notes in Computer Science (including sub-series Lecture Notes in Artificial Intelligence and Lecture Notes in Bioinformatics), vol. 1496, p. 130-137, 1998, which is hereby incorporated by reference in its entirety.

FIG. 3C illustrates a flowchart of an example method 300 for training a deep learning network based on human vascular structures (e.g., training option (3)—training data obtained by from full-quality human clinical angiographic data that have been computationally modified to simulate lower chemical contrast and x-ray doses). In this example, at step 302, a first set of angiographic images of one or more human vascular structures is obtained at standard doses of chemical contrast and x-ray radiation; at step 304, a second set of angiographic images of one or more human vascular structures is obtained which is simulated at reduced doses of chemical contrast and x-ray radiation; and, at step 306, the deep learning network is trained based on the first and second sets.

In one example, anonymized human angiographic data may be obtained at the standard chemical contrast and x-ray doses that are currently used to generate high quality angiographic images. Vascular structures may be extracted from these images by human editing and/or mathematical vessel structure filters. Then, the angiographic images may be computationally modified to simulate lower chemical contrast crossed by lower x-ray doses, e.g., based on computational methods described in M. Elhamiasl and J. Nuyts, “Low-dose x-ray ct simulation from an available higher-dose scan.” Phys Med Biol, vol. 65, no. 13, p. 135010, 2020 Jul. 8, which is hereby incorporated by reference in its entirety.

The simulation of reduced x-ray radiation dose may be generated by adding a mixture of Poisson and Gaussian noise to the data gathered by the x-ray detector. The greater the added noise, the lower the simulated x-ray dose. To simulate lower chemical contrast, an angiographic image may be characterized by a histogram of pixel values. By treating an angiogram as normalized so that greater attenuation of x-rays by the imaged tissue is rendered as greater brightness, when a contrast bolus passes through the vasculature being imaged, the pixel histogram shifts to the right as there are more pixels with brighter values because of the contrast in the imaged blood vessels; accordingly, lower chemical contrast doses may be simulated by applying Poisson and Gaussian noise to the pixels that shift to the right in the histogram as the contrast bolus passes through. For example, a pre-contrast image of the angiographic field may be obtained using a full dosage of x-ray radiation without chemical contrast agent (e.g., before a chemical contrast agent is administered), and a post-contrast angiographic image of the same angiographic field may be obtained using full dosages of a chemical contrast agent and x-ray radiation (e.g., after a chemical contrast agent is administered). For corresponding pixels in the two images, a difference in brightness or intensity between the two corresponding pixels in the images may be determined (e.g., by subtracting their respective pixel values). The brightness difference between the two corresponding pixels may be stored (e.g., as a floating point number between 0 and 1) in a first matrix, and a second matrix of random numbers (e.g., floating point numbers between 0 and 1 randomly generated using a Poisson or Gaussian distribution) may be generated which corresponds in size to the first matrix. For each non-zero cell in the first matrix, the random number in the corresponding cell of the second matrix may be subtracted from the non-zero cell. The results may be stored in a third matrix which may then be added to the pixel values in the pre-contrast image to obtain a simulated reduced-dosage angiographic image.

To avoid registration error due to movement of a vessel in the angiographic field of view before and after administration of the chemical contrast agent, pixels representing the vessel may be identified, and a greater quantity of random noise may added to these pixels to simulate a low dose of chemical contrast agent. In particular, the noise may have a negative mean to simulate a lowering of signal from lower contrast, and a high standard deviation to simulate lowering of signal noise. Using DICOM pixel values of 0-32,000 as an example (instead of floating point numbers between 0 and 1), suppose that the mean pixel value in an image before the chemical contrast agent arrives is a first number (e.g., 50±10). After the chemical contrast agent arrives, some pixels turn brighter because they represent vessels containing the chemical contrast agent. Thus, the mean pixel value increases overall (e.g., to 60±15). In such a case, pixel values greater than the pre-contrast mean value plus two standard deviations (e.g., 50+20) may be assumed to be vessel pixels (i.e., pixels that represent vessels). Thus, a greater quantity of random noise may be added specifically to the vessel pixels to simulate lower contrast dose.

Any suitable data preparation operations may be applied for simulated dose reduction computational transforms. In one example, one or more data augmentation operations may be performed to reduce over-fitting in the deep learning neural network. Examples of such data augmentation operations may include arbitrary translations and rotations of the training data. In one example, the simulated dose reduction transforms may be incorporated into a data augmentation step.

The simulated lower dose images, which preferably have worse signal-to-noise ratios than the unmodified images, may be provided to the neural network. The neural network may, in turn, use the vascular structures obtained from the images taken with standard chemical contrast and x-ray doses as reference vascular data (i.e., a known output) that the network is trained to detect and produce. Thus, the simulated lower dose data may be used as an input to train a deep neural network to produce angiographic images comparable to those obtained from the full chemical contrast and x-ray dose data. In an embodiment, the reference vascular data may include segmented angiographic images obtained at full dosages of chemical contrast and x-ray radiation to train the deep neural network to produce high-quality segmented angiographic images from corresponding non-segmented angiographic images obtained or simulated to have been obtained at reduced dosages of chemical contrast and/or x-ray radiation.

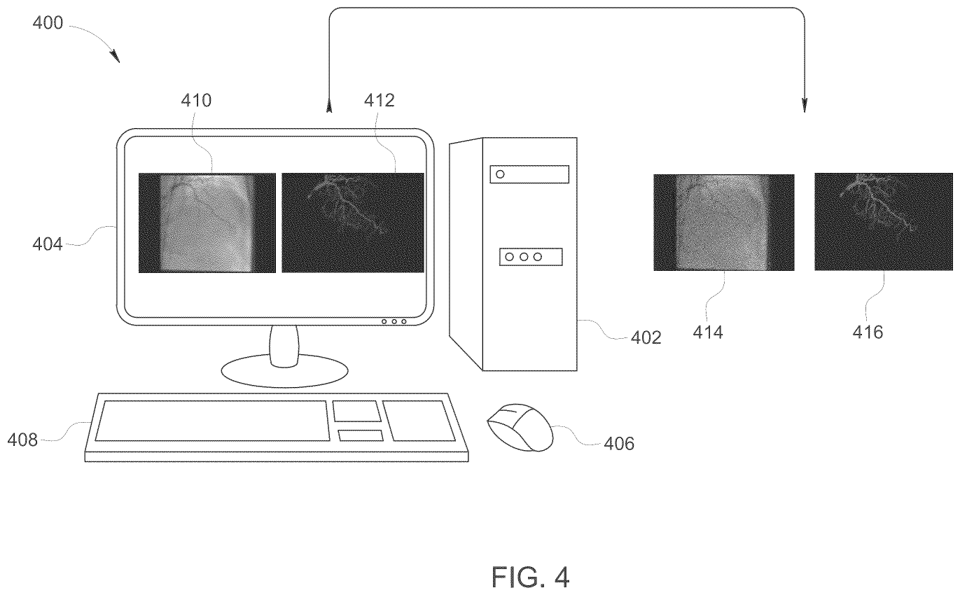

FIG. 4 illustrates an example system 400 configured to generate images of vascular structures simulated at lower doses of chemical contrast and x-ray radiation, according to an example embodiment. The example system 400 includes a computer 402, a display 404 connected to the computer, a pointing device 406 such as a mouse or trackpad connected to the computer, and a keyboard 408 connected to the computer. Computer 402 may include a processor in communication with memory and/or other non-transitory data storage devices containing instructions executable by the processor to segment angiographic images. The computer 402 may also store instructions executable by the processor to operate a neural network in training and/or deployment modes. Computer 402 may also have a communications interface configured to send and receive angiographic data over a communications network such as a local area network or a wide area network or the like. The system 400 may be leveraged for multi-frame deep learning. In one example, a human coronary artery angiogram is obtained. In this example, angiography of the human heart is employed; however, these techniques may apply to angiography of other organs or animals. In the example of FIG. 4, the neural network may be operating in a training mode.

Angiographic images stored on the computer 402 may be displayed on the display 404 of the computer system 400. An example coronary angiogram image 410 is shown being displayed on the display 404. Computer system 400 may obtain image 410 in any conventional manner. For example, the image 410 may be obtained directly from an angiographic imaging system, such as the system shown in FIGS. 1A and 1B, over a hardwired connection, a wireless connection, or a communications network. In another example, computer system 400 may obtain image 410 from a remote source via a local area network, a wide area network, or some other type of communications network. In yet another example, computer system 400 may upload the image from a portable data storage device such as a USB thumbdrive, a DVD, or the like. In one example, a human analyst examines the example image 410 and interacts with the system by employing a graphical user interface device such as a mouse 406 to select (i.e., paint) the pixels that represent blood vessels in a segmented coronary angiogram image 412. In certain examples, segmenting may be performed purely by a human analyst by painting over an angiographic image. In other examples, a mathematical or deep learning segmentation algorithm may make an initial guess at the segmentation. In still other examples, the mathematical or deep learning segmentation algorithm may perform the painting autonomously.

FIG. 4 further illustrates simulated dose reduced coronary angiogram image 414, which has a simulated reduction of x-ray dose compared to the angiographic image 410. The angiographic image 410 may be used to generate a simulated dose reduced coronary angiogram image 414 using any suitable techniques for simulating reduced x-ray radiation doses, as discussed above. For example, computer system 400 may be used to generate the simulated dose reduced coronary angiogram image. Once generated, simulated dose reduced coronary angiogram image 414 may be employed for training of the deep learning neural network by reference to the segmentation of the same angiographic image obtained at full x-ray dose at conserved segmented coronary angiogram image 416 (which may be the same image as segmented image 412).

In FIG. 4, the angiographic image 410 and the segmented image 412 are shown being displayed side by side, but in other examples the painting may take place on the same image on the display 404. Thus, the painting may be overlaid on the angiographic image 410 to generate the segmented image 412.

In training mode, the neural network may obtain, as training data, a plurality of temporally consecutive or contiguous images (e.g., five images) obtained at standard doses and a plurality of corresponding images obtained at (or simulated to have been obtained at) low doses. The images may be selected from a Digital Imaging and Communications in Medicine (DICOM) file including, e.g., approximately eighty total images. Each image may have a size of 512×512 pixels, and each pixel may be represented in DICOM format as an integer (e.g., between approximately 0 and approximately 16,000).

In an embodiment, one or more of the temporally consecutive or contiguous images (e.g., five images) obtained at standard doses may be used as a known output for the neural network, and one or more of the plurality of corresponding images obtained at (or simulated to have been obtained at) low doses may be used to encode the input layer of the neural network. Based on the training data, the neural network may form connections between individual neurons, each connection having one or more associated weights (e.g., floating point numbers) to produce the known output from the input. In an embodiment, if the known output comprises a segmented angiographic image, the neural network may be trained to assign, to each pixel in the output image, a floating point number between 0 and 1 representing the probability of the pixel being part of a blood vessel. In another embodiment, the neural network may assign, to each pixel, a plurality of floating point numbers between 0 and 1, inclusive, wherein each floating point number represents a probability of the pixel being part of a certain feature (e.g., a blood vessel, a catheter, and/or a branch point). For example, three floating point numbers between 0 and 1 may be assigned, wherein one floating point number may represent the probability of the pixel being part of a blood vessel; another floating point number may represent the probability of the pixel being part of a catheter; and another floating point number may represent the probability of the pixel being part of a branch point (e.g., where one blood vessel splits into two or more blood vessels).

A checkpoint file that stores weights for the neural network may be used in a deployment/prediction mode. For example, training with images obtained at standard angiographic doses may result in a first checkpoint file with one set of weights, and training with images obtained at (or simulated to have been obtained at) lower angiographic doses may result in a second checkpoint file with a different set of weights. The first checkpoint file may be applied to an input of one or more images obtained at standard angiographic doses to obtain one or more segmented images, and the second checkpoint file may be applied to an input of one or more images obtained at (or simulated to have been obtained at) lower angiographic doses to obtain one or more segmented images. The second checkpoint file may be applied in a clinical setting where lower angiographic doses are administered to spare the patient adverse side effects of higher angiographic doses while conserving angiographic image quality.

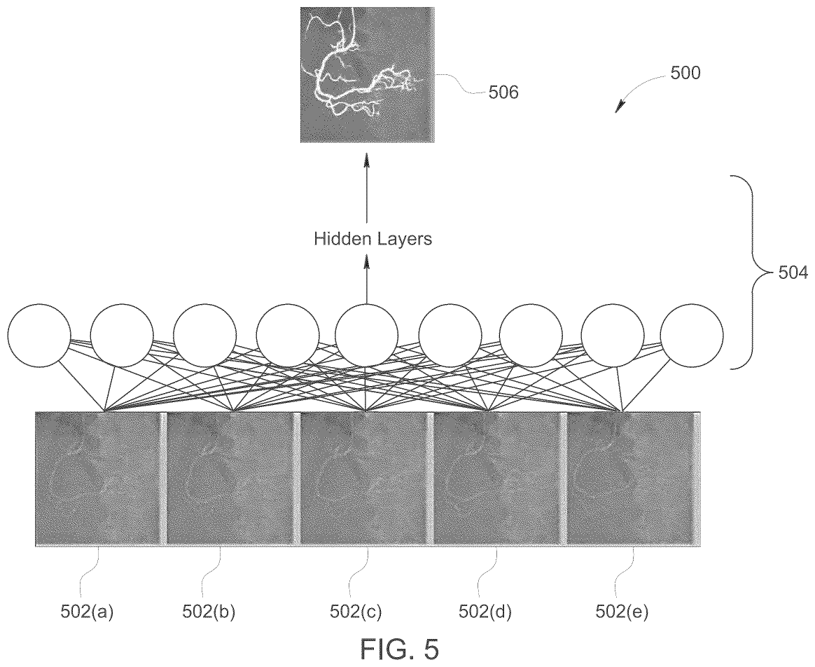

FIG. 5 illustrates an example method 500 for segmenting vasculature objects in a single image from an angiogram acquired at low chemical contrast or x-ray doses. In this example, a convolutional network draws information from several angiographic images to perform the segmentation. In the example of FIG. 5, the neural network may be operating in a deployment mode.

Analyzing (e.g., performing calculations on) an entire sequence of angiographic images may exceed practical computer memory and computational speed limits; thus, as described in connection with FIG. 5, the techniques described herein may enable generation of a high-quality image based on a sub-sequence of images within the relevant computer memory and computational speed constraints. As used herein, the term “sequence” may indicate an entire set of angiographic images, e.g., images that are fluoroscopically acquired across the travel of the injected contrast bolus. The term “sub-sequence” may indicate a subset of the sequence of images that are provided to a deep learning neural network system to estimate the vascular structure. Angiographic images in a “sub-sequence” are preferably temporally contiguous or consecutive, but may be separated by intervening images.

FIG. 5 demonstrates the implementation of the vessel segmentation of a single angiographic image from a sub-sequence of angiographic images that are noisy because they were acquired with low chemical contrast or low x-ray radiation doses. In this example, the angiographic image that is segmented is drawn from a sub-sequence of five temporally adjacent angiographic images 502(a)-(e), which are drawn from physically reduced chemical contrast and x-ray radiation dose angiograms. In this example, the middle (third) image 502(c) in the sub-sequence of five angiographic images is the target image (image of attention). The target image may be the image for which the neural network is to generate a higher-quality version.

The sub-sequence of the five angiographic images 502(a)-(e) may be combined and supplied to a convolutional neural network 504 as a single input. For example, if each image comprises a 512×512 vector of pixel values, the five angiographic images may be inputted or encoded to the neural network as a 5×512×512 vector of pixel values, and the neural network may be trained to produce a single high-quality and/or segmented angiographic image corresponding to one of the five angiographic images (e.g., the middle image 502(c)).

The convolutional neural network 504 may have been previously trained with simulated low-dose, noisy images against vessel segmentations extracted from the standard-dose, non-noisy images (e.g., as discussed above in relation to FIG. 4). For example, to train the neural network to produce a single high-quality and/or segmented angiographic image from five lower-quality temporally contiguous images, a sub-sequence of lower-quality images containing a target image may be obtained. The target image is preferably located in a middle of the sub-sequence so that there are one or more images before the target image and one or more images after the target image. In this way, the neural network may be trained to use spatiotemporal information in providing an output. In the sub-sequence, the number of images before the target image may differ from the number of images after the target image. However, in a preferred embodiment, the target image is centrally located in the sub-sequence so that there are an equal number of images on each side of the target image. For example, in the case of five temporally contiguous images, the target image is preferably located between two temporally contiguous images on each side of the target image. In an embodiment, the neural network may be trained with a plurality of sub-sequences. For example, if there are 10 images from a first angiogram and 12 images from a second angiogram available, a training set may be assembled with up to fourteen sets of unique sub-sequences in which each subsequence consists of five temporally contiguous images. That is, six unique sub-sequences of five temporally contiguous images can be assembled from the first angiogram (because the third through eighth images are each located in the middle of five temporally contiguous images), and eight sub-sequences of five temporally contiguous images can be assembled from the second angiogram (because the third through tenth images are each located in the middle of five temporally contiguous images). The first, second, penultimate, and last images in each series are not targets in this example because they are not located in the middle of five temporally contiguous images. While it has been found that using sub-sequences of five temporally contiguous images is advantageous in that it allows for a high-quality and/or segmented image to be produced with less processing and relatively few images at ends of the sequence not themselves being targets, present techniques may be adapted to use sub-sequences containing fewer than five temporally contiguous images or more than five temporally contiguous images. In general, increasing the number of images in a sub-sequence will tend to increase the resulting signal-to-noise ratio the images produced by the neural network. At the same time, increasing the number of images in a sub-sequence increases the number of images dropped-off at the ends.

While it is advantageous to use an odd number of temporally contiguous images as a sub-sequence for training a neural network to produce a high-quality and/or segmented image corresponding to a target image at the center of the subsequence (e.g., because it is based on an equal amount of spatiotemporal information before and after the target image), the present techniques may be adapted to produce a high-quality and/or segmented image corresponding to a target image that is not at the center of the sub-sequence (e.g., the first, second, fourth, or fifth image of a five-image sub-sequence).

The present techniques may also be adapted to use sub-sequences consisting of an even number of temporally contiguous images. An advantage to using sub-sequences consisting of an even number of temporally contiguous images is that it may permit greater flexibility in the location of the target image and the size of the sub-sequence (e.g., allowing the first or last image in a sequence of images to be targeted using only a sub-sequence of only two temporally contiguous images). It will also be appreciated that the present techniques may be adapted to use a combination of odd and even-numbered sub-sequences to produce high-quality and/or segmented images corresponding to corresponding targets located anywhere in the sequence. In another embodiment, a plurality of neural networks may be deployed in which each neural network is trained with a different number of input images, ranging from as few as one image up to as many images as processing resources allow. The use of a plurality of neural networks in this manner allows high-quality and/or segmented images to be produced from every image in an angiogram.

In a preferred embodiment, the target image is the middle of five temporally contiguous images, where acquiring images temporally before and after increases the expected concordance in the estimate of the middle image. In other embodiments, the temporal sequencing may be altered to promote real time behavior. In an embodiment, the target image is the most recent of three images. This lag between the moment of acquisition of the third image and the generation by the machine learning system of its transformed version corresponds the perceptual lag to the human user. In this embodiment there may be autoregressive feedback to the machine learning system, where in addition to the prior three images being input to the machine learning system, one or more of the associated prior predicted images are input as well.

In an embodiment, images in a training set may be subjected to a data augmentation step in which, e.g., images are translated and/or rotated prior to being inputted to train the neural network to generalize better. It will be appreciated that a simulation of reduced dosages of chemical contrast agent and/or x-ray radiation may be incorporated as part of the data augmentation step.

Because it is operating on the sub-sequence of five noisy angiographic images 502(a)-(e), the trained convolutional neural network 504 may estimate a single segmented image 506 that has a greater signal-to-noise ratio than if the trained convolutional neural network were operating on only a single noisy angiographic image. The segmented image 506 may represent the deep learning neural network estimation of the vascular structure of the middle (third) image in the sub-sequence of five noisy angiographic images 5(a)-(e).

More specifically, the neural network 504 may load the floating point numbers from the checkpoint file generated during the training mode, and, based on the floating point numbers, output the segmented image 506 from the five noisy angiographic images 5(a)-(e). The neural network may generate, as output, one or more of three images: one image based on the probability of pixels being part of a blood vessel; another image based on the probability of the pixels being part of a catheter; and another image based on the probability of the pixel being part of a branch point. These three images may be stacked/overlaid to generate the segmented image 506 showing blood vessels, a catheter, and/or branch points in high-quality, e.g., as if the target image was obtained at standard angiographic doses.

In one example, the convolutional neural network 504 used for angiographic segmentation may be based on a U-Net architecture, e.g., as described in O. Ronneberger, P. Fischer, and T. Brox, “U-net: Convolutional networks for biomedical image segmentation,” in Medical Image Computing and Computer-Assisted Intervention—MICCAI 2015, N. Navab, J. Hornegger, W. M. Wells, and A. F. Frangi, Eds. Cham: Springer International Publishing, 2015, pp. 234-241, which is hereby incorporated by reference in its entirety.

In one specific example, source code for the deep learning neural networks described herein may be written in Python language and generated as a U-net structure in the Pytorch machine learning software library (https://pytorch.org, which is hereby incorporated by reference in its entirety). The U-net structure may be three-dimensional in the sense that each image has two spatial dimensions, and the vasculature within the images may be estimated from both the image and temporally nearby angiographic images. In one specific example, the Pytorch library for neural network machine learning may include a Python base class named nn.Module, as documented at https://pytorch.org/docs/stable/generated/torch.nn.Module.html, which is hereby incorporated by reference in its entirety. All neural network modules may comprise subclass nn.Module. A python class named UNet may inherit from nn.Module. The Python class Unet may include the convolutional neural net structure with spatiotemporal properties. It will be appreciated that the techniques described herein may be implemented using any suitable machine learning mechanism.

It will be appreciated that any suitable programming languages, libraries, toolsets, and/or other mechanisms may be used for developing a deep learning neural network in accordance with the examples provided herein. For example, the U-net structure may be extended to a full three-dimensional structure that simultaneously estimates the vascular structures in the plurality of the temporally adjacent angiographic images.

The convolutional neural network 504 may have the structure of an encoder-decoder. The convolutional neural network 504 may also include jump connections between layers of the same size on the encoder and the decoder. These jump connections may enable the output of segmented image 506 with a similar degree of granularity as the angiogram sub-sequence inputs (e.g., the sub-sequence of the five angiographic images 502(a)-(e)). The loss function used to train this architecture may be a linear combination of a classification loss-function, cross-entropy, and a dice loss function for crisp boundary detection.

To prevent the movement of blood vessels between angiographic image frames from interfering with the angiographic data, the structure of convolutional neural network 504 may have a high spatiotemporal convolutional density. That is, an organ that experiences larger motion, such as the heart, may be imaged at 15 Hz, with a neighborhood of five images being used to determine the vasculature present in the middle image; an organ with less motion, such as the brain, may be imaged at, e.g., 6 Hz, with a neighborhood of five images being used to determine the vasculature present in the middle image. Thus, the convolutional neural network 504 may account for both lesser motion (such as motion in the brain, where motion is limited by the surrounding rigid container of the cranial bone) as well as greater motion (such as motion in the heart, which is a muscular organ that is continually beating to pump blood into an arterial system).

In a deep learning network training mode (in contrast to the deployment mode illustrated in FIG. 5), the roles of the data sources (inputs) and products (outputs) may be altered. For example, the image 506 may represent a ground truth representation of the vasculature structure as would be obtained from full dose chemical contrast and x-ray radiation dose angiography, and the one or more images of the sub-sequence 502(a)-(e) may be obtained from empirically reduced dose studies from animal or physical organoid model angiography, or computationally simulated reduced images drawn from the ground truth full dose image 506. In training mode, training may occur based on both the one or more images of the sub-sequence 502(a)-(e) and the segmented image 506.

While FIG. 5 illustrates the sub-sequence 502(a)-(e) as having five angiographic images, it will be appreciated that the quantity of five is simply an example. In some circumstances, fewer or greater than five images may be employed, even within the same angiographic study. For example, more than five angiographic images may be used to estimate the segmentation of the middle image of an angiographic sequence containing dozens of individual images. This may increase the signal-to-noise ratio in the angiographic image of interest compared to using only five angiographic images (but at the cost of increased computational resources), thereby allowing for further reduction in chemical contrast and/or x-ray radiation doses.

In such circumstances, the vessel segmentation of angiographic images toward the beginning or the end of the sequence may be estimated based on fewer surrounding images. For example, the sixth image may be segmented based on its position in the middle of five images (e.g., numbers 4, 5, 6, 7, and 8), whereas the fifth image cannot be in the middle of a sub-sequence of five images, but could be treated as being in the middle of a sub-sequence of three images (e.g., numbers 4, 5, and 6).

In addition, there may be circumstances where the angiographic image of attention (e.g., the target image) is not in the middle of the sub-sequence, but is instead in another position (e.g., near the beginning or the end of the sub-sequence). For example, during angiographic catheter positioning maneuvers, the angiography physician may choose to perform intermittent real-time angiographic contrast study injections while acquiring fluoroscopic images. The angiographic image of interest cannot be a middle image in the context of real-time angiographic imaging because the future angiographic images have not yet been acquired. Instead, a deep learning network may perform the signal to noise enhancement based on the target image and a temporally preceding plurality of images.

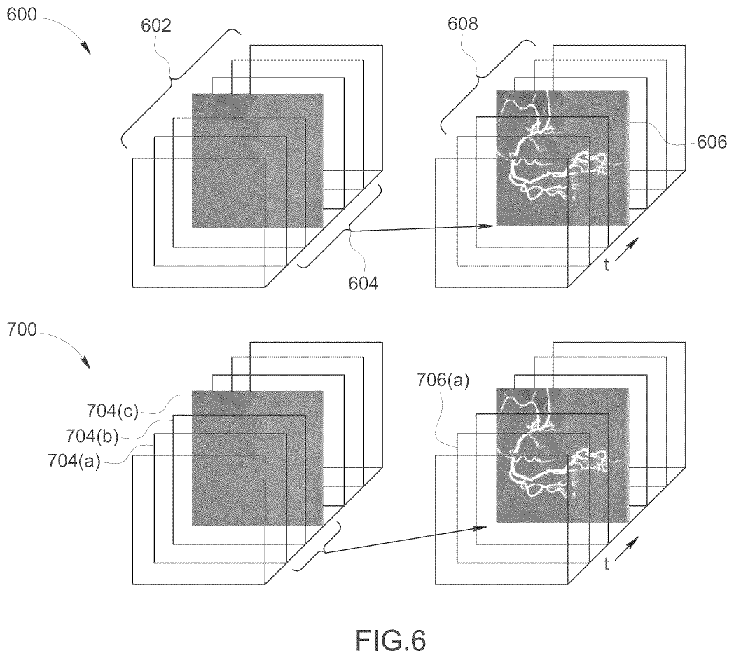

FIG. 6 depicts the sub-setting of a sequence of angiographic images into a plurality of overlapping sub-sequences where each estimates the vascular structure for one angiographic image. More specifically, FIG. 6 illustrates a first example method for estimating a higher-quality image in which a target image is in the middle of a sub-sequence, and a second example method for estimating a higher-quality image in which a target image that is not in the middle of a sub-sequence. The first method may apply to offline fluoroscopic angiography, and the second method may apply to real-time fluoroscopic angiography.

The first method 600 is illustrated in the top panel of FIG. 6. As shown, a sequence of angiographic images 602, of total length n, is produced. Within the sequence of angiographic images 602, there is a sub-sequence of five angiographic images 604 of length five where the image of attention for segmentation by the deep learning network is in the middle. The neural network may produce a segmented image 606 that corresponds to the middle (third) angiographic image in the sub-sequence. This process may be incremented by one image for the sequence of angiographic images 602 until every image but the first two and the last two have been the image of attention for deep learning segmentation. This may produce a sequence of segmented images 608.

The second method 700 is illustrated in the bottom panel of FIG. 6. Here, the angiographic image of attention may be the first (most recent) angiographic image 704(a) of a sub-sequence of angiographic images 704(a)-(c). The neural network may apply a deep learning calculation to improve the signal-to-noise ratio of the target image 704(a) to produce a high-quality segmented version 706(a) of the target image, and thereby to improve the tolerance of the image quality to reductions in chemical contrast dose and x-ray radiation dose. For example, the neural network may use the target image and one or more subsequent, temporally contiguous images as the inputs for the deep learning network calculation.

In another aspect of the disclosure, a method of training a machine learning system to facilitate real time adjustment of a chemical contrast agent dosage and/or an x-ray radiation dosage during angiographic imaging is disclosed. A first machine learning model may be provided inputs via a processor of a first set of one or more angiographic images obtained using a first chemical contrast agent dosage and a first x-ray radiation dosage. The first machine learning model may also be provided, as input, a concordance metric indicating a degree of similarity between an estimated segmentation of the first set of one or more angiographic images generated by a second machine learning model (e.g., a machine learning model trained to segment angiographic images as described previously herein) and one or more segmented benchmark angiographic images provided as inputs to the second machine learning model. The segmented benchmark angiographic images may be obtained using a second chemical contrast agent dosage or a second x-ray radiation dosage that is higher than the first chemical contrast agent dosage or the first x-ray radiation dosage. The method may generate, as an output of the first machine learning model an estimated concordance metric for the first set of one or more angiographic images. The method may compare the estimated concordance metric and the inputted concordance metric and may perform a back propagating step to adjust parameters of the first machine learning model based on the comparing step.

In one embodiment, the disclosed method may provide, as an input to the first machine learning model, via a processor, a dosage value based on the first chemical contrast agent dosage or the first x-ray radiation dosage used to obtain the first set of one or more angiographic images.

In one embodiment, the first and second machine learning models may be separate machine learning models. One of the machine learning models estimates a segmented image and the other machine learning model separately provides a metric for estimating the error of the segmentation. A real-valued metric suitable for estimating the error of the segmentation is the Dice coefficient; however, other metrics of segmented image quality may be employed. While two separate machine learning models are depicted, it will be appreciated that the two machine learning models may be a single, unified machine learning model.

In an embodiment, different concordance metrics may be used to train the first machine learning model and the second machine learning model. In one embodiment, the concordance metric may be a loss metric. In one embodiment, the concordance metric may include a loss metric slope. In one embodiment, the concordance metric may be a cross-entropy metric. In one embodiment, the concordance metric may include a cross-entropy slope.

In one embodiment, the concordance metric may be a Dice coefficient. The Dice coefficient is a measure of the goodness of a segmentation. A task in the analysis of an angiographic image is the segmentation of blood vessels. This means the identification of those pixels in the image that represent portions of a blood vessel, as defined by containing angiographic contrast. Let X be the set of pixels segmented as belonging to blood vessels by the machine learning neural network. Let Y be the set of pixels belonging to blood vessels as per the gold standard image. The Dice coefficient is defined as

$$D ( X , Y ) = \frac{2 \vert X ⋂ X \vert}{\vert X \vert + \vert Y \vert}$$

where the vertical bars ∥ indicate cardinality. For example, |X| is the number of pixels belonging to set X. The Dice coefficient ranges from 0 to 1. Larger numbers reflect better performance of the machine learning segmentation. In this embodiment, the Dice coefficient is employed as a measure of the segmentation and image quality produced by the machine learning neural network. Other measures of segmentation and image quality could be employed and remain consistent with the spirit of this invention.

The Dice coefficient serves as a proxy for a threshold of image quality needed to satisfy a decision-making clinical need in an angiogram setting. The goal is to deliver the least x-ray radiation consistent with satisfying that decision-making criteria. The higher radiation dose delivered, the higher the signal to noise ratio and the better the image statistics will be. This is more likely to satisfy the clinical decision-making need, at the expense of higher radiation dosage to the patient.

The disclosed method employs a machine learning model to produce the best possible image in a spatial temporal sense, but also to estimate the image quality metric (e.g., a concordance metric) and feed that back into the angiographic imaging machine and/or an autoinjector in real time to find the minimal x-ray dose and/or chemical contrast dose that satisfies the need. The disclosed machine learning model learns the image quality metric in real time—e.g., the Dice coefficient. In an example embodiment, the Dice coefficient is the fraction of pixels that the machine learning model designates as a heart vessel.

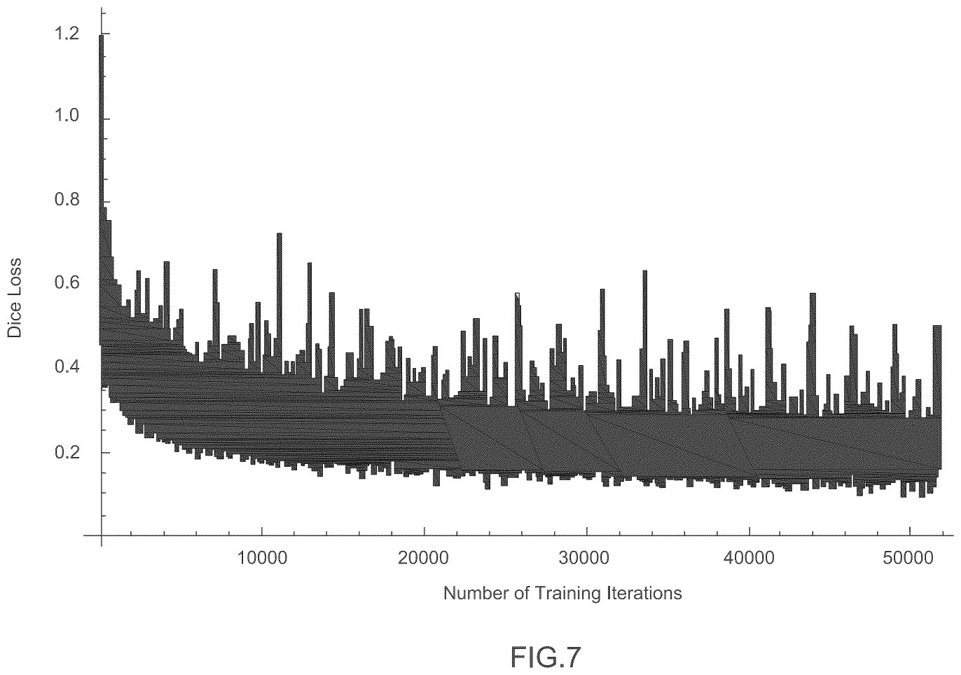

FIG. 7 is a plot of Dice loss versus number of machine learning model training iterations. The plot illustrates example Dice coefficient data variability during training of a machine learning model with angiographic images. The Dice coefficient may vary from iteration to iteration. As training progresses, the model with the best Dice coefficient is the one that is produced at the end, not necessarily the model from the final training iteration.

As formatted, the vertical or y axis of the plot is “Dice loss”, which is equal to 1-Dice, and the horizontal or x axis is the number of training iterations of the machine learning model. From the plot, one can understand that a Dice of about 0.9 equals a Dice loss of 0.1. The tradeoff between dose reduction and a good-enough Dice coefficient may be evaluated from iterations of the training model.

The inventors have discovered that lower simulated x-ray doses that give a Dice coefficient of about 0.7 may be considered less than satisfactory, a Dice coefficient of about 0.8 may be considered acceptable, and a Dice coefficient of 0.9 may be considered good. The loss in Dice is simulated from overdose exposure.

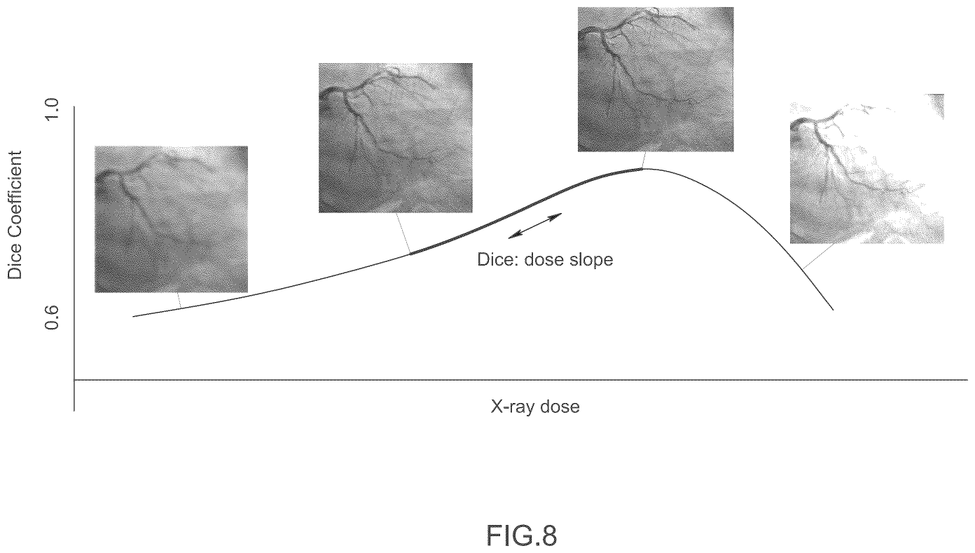

FIG. 8 illustrates a schematic comparison of example image segmentations obtained with different x-ray doses. The example image segmentations are superimposed on a plot of Dice coefficient verses x-ray dose, wherein the image segmentations correspond to different Dice coefficients and Dice coefficient slopes. The value of the Dice coefficient ranges between 0 (not similar) to 1 (very similar). A sufficient threshold Dice coefficient for adequate machine learning model segmentation compared to the gold standard human coded segmentation may be determined by evaluation of testing images fed into the machine learning model. A sufficient number of testing images and trials are performed to determine a threshold acceptable Dice coefficient for a given angiographic machine or machine parameters. For example, a Dice coefficient of about 0.8 may be considered acceptable, and a Dice coefficient of 0.9 may be considered good.

As discussed above, other concordance metrics and values may be used. For example, another metric that may be used in training machine learning models is the cross-entropy. Other suitable image quality metrics with a discoverable relationship to x-ray dose that can be exploited for clinical benefit and hence be used as a concordance metric.

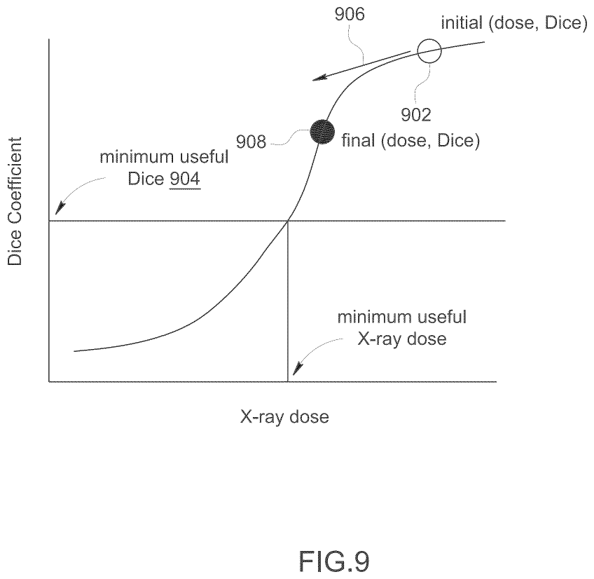

FIG. 9 shows an example Dice coefficient versus x-ray dose curve that may be used to adjust x-ray dose. When a Dice coefficient is calculated in the training phase or inferred in an execution phase, it is compared to the minimum Dice coefficient that prior studies have shown to be consistent with the image and segmentation quality needed to achieve the diagnostic and treatment goal of the angiographic study (e.g., a minimum Dice coefficient of 0.8). Dice coefficients and x-ray doses form coordinate pairs. In the example shown, the initial X-ray dose to Dice coordinate pair 902 is well above the required Dice threshold 904 for clinical use. The x-ray dose may be reduced as per the Dice coefficient to X-ray dose slope 906 to yield a new adjusted X-ray dose to Dice coordinate 908 that satisfies the clinical need at a lower x-ray dose. On the other hand, if the Dice coefficient is too low to satisfy the clinical need, then knowledge of the curve can be used to estimate the minimal x-ray increase needed.

The curve in FIG. 9 may be estimated from archival human and animal data, or, as experience and data accumulate, it may be encoded into the weights of the neural network and be inferred in real-time in the execution phase by the disclosed machine learning model.

In clinical use of the disclosed method, an initial Dice coefficient may be calculated by the disclosed machine learning model. For example, an initial Dice coefficient slope may be calculated by dividing the Dice coefficient by the x-ray dose. The initial Dice coefficient slope may then be used to adjust the x-ray dose for the next iteration of images. A goal of the disclosed method is to reduce the x-ray dosage to generate an equivalent diagnostic image as a gold standard segmentation image. However, there is a minimum useful Dice coefficient that is acceptable for accurate image diagnostic decision-making criteria, as well as a minimum useful x-ray dosage to generate a diagnostically useful angiogram. FIG. 9 illustrates how the Dice coefficient slope may be used for diagnostic decision-making.

The Dice coefficient plays a prominent role in the actual learning as a loss function that is used to train against. In the training stage, a machine learning model is given a data sample set and it generates a prediction of which of the examined pixels are vessels and then that prediction is compared to a human gold standard annotation of the image. The difference between these two is used to back-propagate to the coefficients of the machine learning model connections. Other loss functions may be implemented as known to one of skill in the art, such as cross entropy mean squared error or other loss functions.

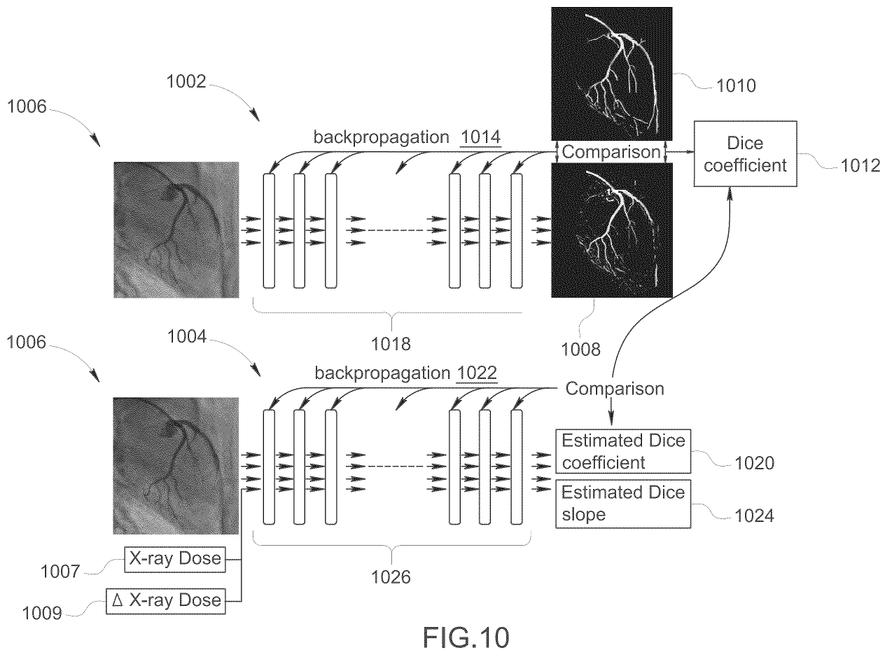

FIG. 10 illustrates an example training mode or process for two machine learning models 1002 and 1004. The first machine learning model 1002, depicted in the upper region of FIG. 10, is trained for clinical use to predict segmented angiographic images from unsegmented angiographic images. That is, the upper-displayed machine learning model 1002 takes as an input an unsegmented angiographic image 1006 and generates a segmented prediction 1008 which may be compared to a gold standard segmented angiographic image 1010 (typically a human hand-coded estimate) based on the same unsegmented angiographic image. From a difference between the segmented prediction 1008 and the human hand-coded estimate 1010, the machine learning model 1002 calculates a concordance metric 1012, such as a Dice coefficient. The machine learning model 1002 then performs a back propagation step 1014 along the machine learning model to improve the prediction. While only one image is depicted in FIG. 10, in practice there will typically be a cine sequence of angiographic images or frames (e.g., typically taken at 15 frames per second) in an angiogram.

More specifically, angiographic image 1006 may be sent as pixels through an image encoder into a plurality of neural network layers 1018 of the machine learning model 1002. The pixels flow through the layers and are decoded as an inferred or predicted segmented angiographic image 1008. The predicted segmented angiographic image 1008 is compared to the gold standard segmented angiographic image 1010, and the difference between them is summarized as a concordance metric 1012, such as a Dice coefficient. The segmented angiographic image 1008 is further compared pixel by pixel with the gold standard segmented angiographic image 1010. The differences may be backpropagated at 1014 through the neural network layers 1018 of machine learning model 1002 to improve the segmentation performance of the machine learning model.

The second machine learning model 1004, depicted in the lower region of FIG. 10, takes the same unsegmented image 1006 (or the same spatial temporal set up images) as used in the first machine learning model and predicts an estimated concordance metric 1020 for a hypothetical segmented angiogram image generated by the upper-displayed machine learning model 1002. That is, the second machine learning 1004 model predicts the concordance metric 1020 that would be calculated if the upper-displayed machine learning model 1004 were to generate a predicted segmented angiographic image from the unsegmented image 1006. The predicted concordance metric 1020, such as the Dice coefficient, is compared to the first machine learning model-generated concordance metric 1012, and then the difference may be back-propagated at 1022 through the neural network layers 1026 of machine learning model 1004 to improve the accuracy of the concordance metric prediction (see, e.g., FIG. 7). The second machine learning model 1004 may also be trained to estimate a concordance metric slope 1024, such as a Dice coefficient slope or Dice versus x-ray dose, in a similar manner.

For example, in order to train the second machine learning model 1004 to predict a Dice coefficient, the unsegmented angiographic image 1006 may be inputted into the second machine learning model 1004 with a Dice coefficient 1012 that was calculated for the segmented image predicted by the first machine learning model 1002 based on the unsegmented image. In an embodiment, the second machine learning model 1004 may also be fed with x-ray dose 1007 and x-ray dose change 1009 for encoding into the layers 1026 of its neural network. The X-ray dose may be obtained in real-time from the fluoroscopic machine that generates the X-rays that drive the imaging. For most angiographic image frames, the X-ray dose does not change. For most frames, then, the value of the X-ray dose change is zero. The estimated Dice coefficient 1020 and estimated Dice coefficient to x-ray dose slope 1024 may be compared to the empiric versions to generate a Dice backpropagation 1022 to train the second machine learning model 1004 to more accurately predict these metrics.

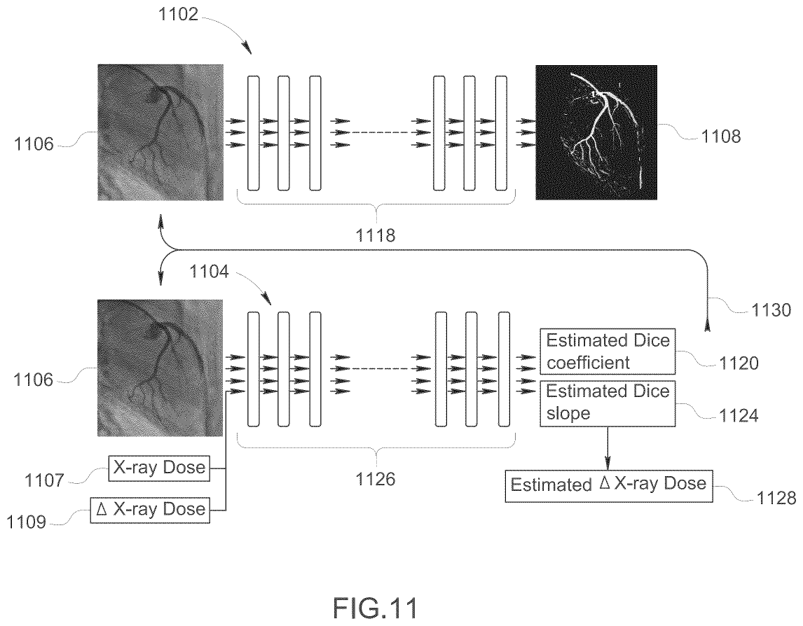

FIG. 11 illustrates an example execution mode for two machine learning models 1102 and 1104. In an embodiment, machine learning models 1102 and 1104 may be trained versions of machine learning models 1002 and 1004, respectively. Comparing the execution mode in FIG. 11 to the training mode in FIG. 10 above, it can be seen that one difference between the two modes is that there is forward inference but no backpropagation in the execution mode. Furthermore, in the example execution mode shown, an angiographic image 1106 is fed into the first machine learning model 1102 as an input, and the same angiographic image 1106 is fed into the second machine learning model 1104 as an input, along with the x-ray dose 1107 used to obtain the angiographic image and the x-ray dose change 1109. In the first machine learning model 1102, the neural network layers 1118 generate an estimated segmented angiographic image 1108. The second machine learning model 1104 infers an estimated Dice coefficient 1120 and an estimated Dice coefficient to x-ray dose slope 1124. Knowledge of the relationship between x-ray dose and Dice coefficient, leads to an estimated x-ray dose adjustment 1128 to achieve a minimum x-ray dosage that satisfies the clinical need. For example, the estimated x-ray dose adjustment 1128 from the second machine learning model 1104 may be communicated to the control mechanism 60 of the angiographic imaging device 28 to cause the control mechanism to adjust the dosage of x-ray radiation administered by the x-ray source to the patient in a subsequent angiographic image or frame.

In an embodiment, the first machine learning model 1102 illustrated in FIG. 11 may accept images outputed by an angiographic imaging device (such as rotational x-ray system 28) in real time. The first machine learning model 1102 feeds the images (e.g., image 1106) through its neural network layers 1118 and produces an estimated or predicted segmented image 1108 based on the unsegmented image 1106 from the imaging device. Meanwhile, the same empirical data (i.e., the same unsegmented image 1106) and corresponding x-ray dose 1107 and x-ray dose adjustment (if any) that was applied to obtain the empirical data goes to the second machine learning model 1104 in the lower portion of FIG. 11 and is fed into its neural network layers 1126. The second machine learning model 1104 produces an estimated concordance metric, such as a Dice coefficient 1120, and an estimated concordance metric slope, such as a Dice coefficient slope 1124. The estimated concordance metric slope, such as the Dice coefficient slope 1124, may be used in real time by the system to adjust the x-ray dose for subsequent imaging (e.g., as shown at 1130). The adjustment may be made manually by an operator of the angiographic imaging system or automatically via a control mechanism of the angiographic imaging system.

It will be appreciated that the first and second machine learning models described above can be adapted to facilitate real time adjustment of the amount of chemical contrast agent administered to a human subject manually or via an autoinjector during an angiographic procedure. For example, instead of (or in addition to) feeding the x-ray dose and x-ray dose adjustment into the second machine learning model, the chemical contrast agent dose and chemical contrast agent dose adjustment used to obtain the unsegmented image can be fed into the second machine learning model. The second machine learning model may then estimate a concordance metric and estimated concordance metric slope, as before, which can be used to adjust the amount of contrast agent administered to the subject for the next image.

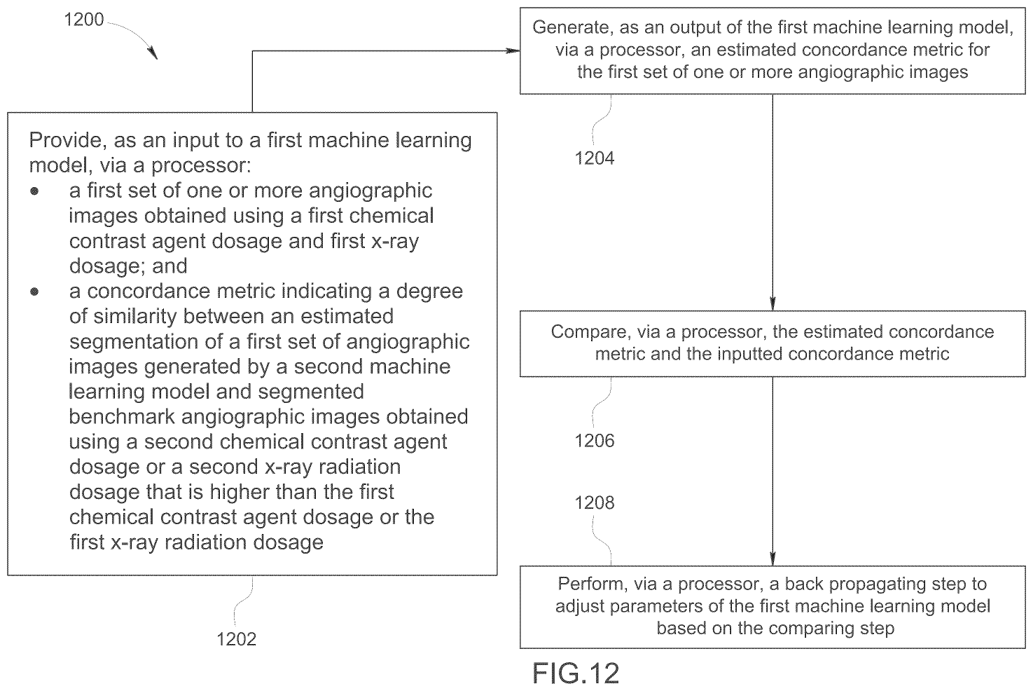

FIG. 12 is a flowchart illustrating a method 1200 of training a machine learning system to facilitate real time adjustment of a chemical contrast agent dosage and/or an x-ray radiation dosage during angiographic imaging according to an example embodiment.

In step 1202, two inputs are provided, via a processor, to a first machine learning model. One of the inputs is a first set of one or more angiographic images from an angiographic imaging device obtained using a first chemical contrast agent dosage and a first x-ray dosage. The other input is a concordance metric indicating a degree of similarity between an estimated segmentation of the first set of angiographic images generated by a second machine learning model and segmented benchmark angiographic images obtained using a second chemical contrast agent dosage or a second x-ray radiation dosage that is higher than the first chemical contrast agent dosage or the first x-ray radiation dosage, respectively.

In step 1204, an estimated concordance metric for the first set of one or more angiographic images is generated, via a processor, as an output of the first machine learning model.

In step 1206, a comparison is made, via a processor, between the estimated concordance metric and the inputted concordance metric.

In step 1208, a back propagating step is performed, via a processor, to adjust parameters of the first machine learning model based on the comparing step.

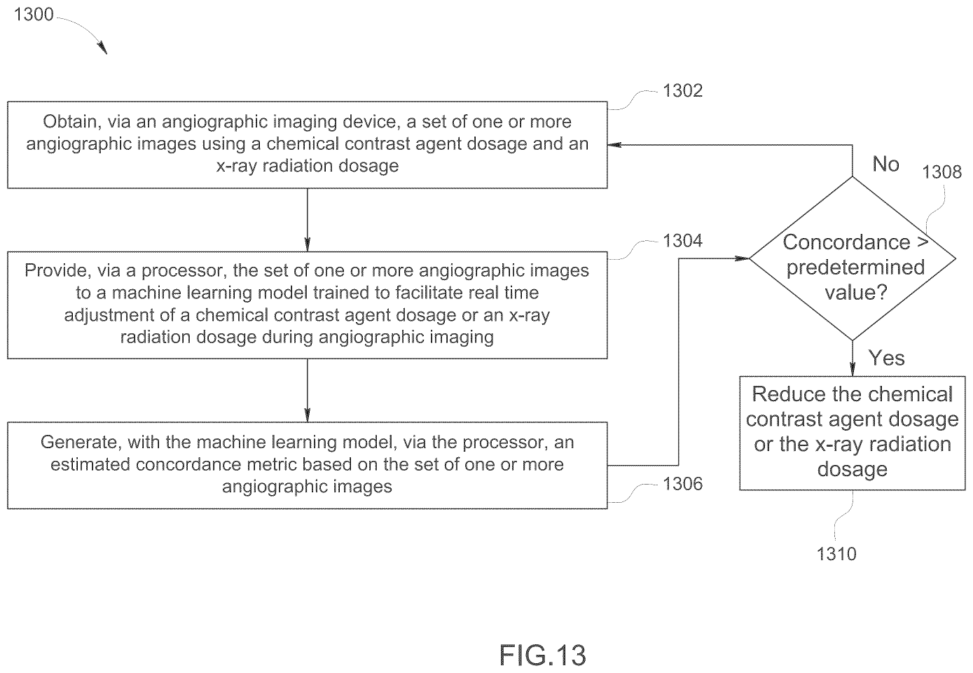

FIG. 13 is a flowchart illustrating a method 1300 of acquiring angiographic images with real time dose adjustment according to an example embodiment.

In step 1302, a set of one or more angiographic images is acquired, via a processor, wherein the images were obtained by an angiographic imaging device using a first chemical contrast agent dosage and a first x-ray radiation dosage.

In step 1304, the set of one or more angiographic images is provided to a machine learning model trained to facilitate real time adjustment of a chemical contrast agent dosage and/or an x-ray radiation dosage during angiographic imaging.

In step 1306, an estimated concordance metric based on the set of one or more angiographic images is generated, via a processor, with the machine learning model.

In step 1308, a determination is made whether the estimated concordance metric is greater than a predetermined value. In an embodiment, the predetermined value is a minimum value to obtain diagnostically useful images. For example, if the concordance metric is a Dice coefficient, the predetermined value may be 0.8.

If the estimated concordance metric is greater than the predetermined value (or in some embodiment greater than or equal to the predetermined value), then the chemical contrast agent dosage and/or the x-ray radiation dosage is reduced to a third chemical contrast dosage or a third x-ray radiation dosage at step 1310. In an embodiment, the step of reducing the dosage may be accomplished by sending an estimated dosage adjustment to an operator of the angiographic imaging device so that the dosage may be manually adjusted or sending a dosage adjustment command to a controller that controls a level of the x-ray radiation source of the angiographic imaging device and/or operation of an autoinjector so that the dosage may be automatically adjusted based on the estimated dosage adjustment.

If the estimated concordance metric is not greater than the predetermined value, then the method may go back to step 1302 such that the next image is obtained using the same dosage as the last image. In an embodiment, if the estimated concordance metric is very low, the system may estimate an increased chemical contrast agent dosage or x-ray radiation dosage that may be administered to improve the quality of a subsequent image.

In one embodiment, the method may include generating, via a processor, a suggested adjustment to the current chemical contrast dosage and/or the current x-ray radiation dosage based on the generated concordance metric.

In one embodiment, the method may include obtaining, via the angiographic imaging device, a subsequent set of one or more angiographic images using a new chemical contrast dosage and/or a new x-ray radiation dosage based on the generated concordance metric, wherein the new dosage is different than the prior dosage.

In one embodiment, the method may include providing, as an input to a second machine learning model, via a processor, the new set of one or more angiographic images. In one embodiment, the method may include generating, with the second machine learning model, via a processor, an estimated segmentation of the new set of one or more angiographic images.

In one embodiment, the method may include adjusting, via a processor, an x-ray radiation dosage emitted by the angiographic imaging device based on the generated concordance metric.

In one embodiment, the method may include adjusting, via a processor, a chemical contrast agent dosage administered by an autoinjector based on the generated concordance metric.

In one embodiment, the method may include comparing the concordance metric with a predetermined value. In one embodiment, the predetermined value is a minimum acceptable probability of concordance. In one embodiment, the method may include reducing the chemical contrast agent dosage or the x-ray radiation dosage if the concordance metric is greater than or equal to the minimum acceptable probability of concordance.

In one embodiment, the concordance metric is determined for an individual angiographic image frame and used to adjust dosage for a subsequent angiographic image frame. In one embodiment, the concordance metric is determined for a plurality of angiographic image frames and used to adjust dosage for a subsequent plurality of angiographic image frames.

In one embodiment, the method may include communicating the suggested dosage adjustment to a health care provider. As an applied example, the disclosed system and method may be used on a human subject undergoing an angiogram. In typical treatment, a chemical contrast solution is injected into the patient. A series of images are generated by the fluoroscopic image tool, in real-time, at a frame rate of 15 frames per second.

As the images are output by the fluoroscopic image tool, the angiogram operator evaluates the images with the given x-ray dosage applied to the patient at the beginning of the examination. A benefit of the disclosed trained machine learning model is that the concordance metric, such as the Dice coefficient, can serve as a proxy to deliver equivalent image quality as a useful diagnostic measure with a lower x-ray dosage.

In one embodiment, the disclosed machine learning model estimates the concordance metric and concordance metric slope, such as the Dice coefficient and the Dice coefficient slope, in real-time. In one embodiment, the concordance metric slope, such as the Dice coefficient slope, is used to adjust the x-ray dose for the next image in the sequence.

In an embodiment, the disclosed machine learning model may be implemented with a feedback mechanism to reduce x-ray dosage by adjusting the frame rate of the fluoroscopic image tool. In one embodiment, the concordance metric, such as the Dice coefficient, may be used as a quality metric to adjust x-ray dosage only when acquiring a frame. As the frame rate is adjusted based on the concordance metric, the quality of the image may be reduced or become more choppy, but the image may still be diagnostically useful with a reduced x-ray dosage to the patient and attending staff.

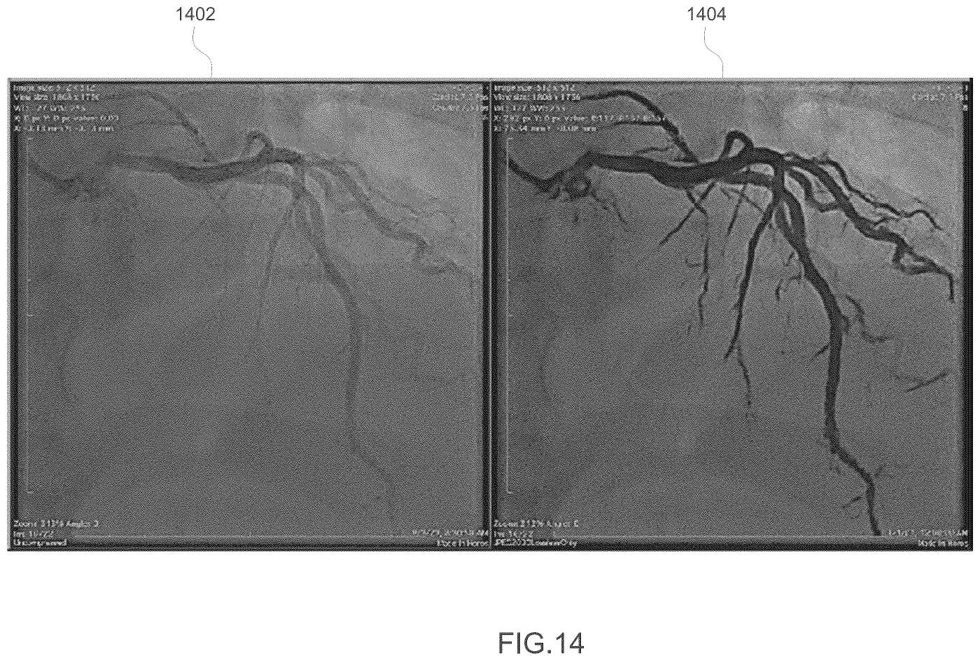

FIG. 14 shows a comparison of two angiographic images 1402 and 1404 obtained with a commercially available angiographic imaging system from Philips. The angiographic image 1402 on the left was captured by operating the angiographic imaging system in the “Fluoroscopy Low” mode without performing any additional imaging processing. The “Fluoroscopy Low” mode delivers 10% of the radiation dose to the patient than is traditionally delivered when capturing a traditional diagnostic angiogram under “Cine” mode, which is the setting normally used by health care professionals to capture an angiogram for diagnostic purposes (e.g., to diagnose diseases of the heart and other organs). The “Fluoroscopy Low” mode on the other hand is typically used for tasks such as interactive catheter manipulation and guidance.

The angiographic image 1404 on the right was captured by the same angiographic imaging system in the “Fluoroscopy Low” mode but was processed using a machine learning model trained to produce segmented angiographic images according to the present disclosure. It can be seen that the image on the right is much better than the image on the left. In fact, the image on the right is as good or better than a normal diagnostic angiogram which would normally be captured using the much higher “Cine” mode radiation settings on the Philips angiographic imaging system.

In one embodiment, an angiographic imaging system is provided with all the electronic components necessary to provide real-time deep learning image processing. These components may include an electronic x-ray detector or capture unit to capture the x-ray photons and convert them into electrical signals in a matrix for image production, an integrated computing unit to apply a deep learning transform to the data, and associated with this a deep learning system for estimating an image quality metric such as a Dice coefficient. The Dice coefficient may then be used in real time to adjust the x-ray dose in order to satisfy the signal-to-noise relationships needed for the clinical diagnostic purpose of the angiogram.

Early angiographic imaging systems included a mechanical device that, in a “cine” or “dynamic” mode, sequentially flipped from one physical x-ray film to the next to obtain a series of angiographic images using a normal dose of x-ray radiation. The images obtained in cine mode were suitable for diagnostic purposes and were recorded for the subject's permanent radiological record. There was also another mode called “fluoro” that administered a lower dose of x-ray radiation but was not intended to be recorded for the permanent record. Instead, the lower fluoro dose was intended for interactive catheter manipulation and guidance but not for capturing angiograms to be part of the permanent radiological record.

In contemporary angiography, there is a graphical user interface that employs traditional terminology, such as cine mode and fluoro mode, but applies it to modes of operation of electronic components of the angiographic imaging system. These electronic components may include an image intensifier, which captures the x-ray data in two spatial dimensions and transforms each element into a gray level in an image that reflects the x-ray arrival at that pixel at a given frame coordinate in time.

While the conventional settings on a graphical user interface for angiographic images of diagnostic quality for the permanent record rely on the traditional grouping of settings known as dynamic or as cine angiography, the grouping of settings known as fluoro is intended to give lower x-ray dose settings not traditionally understood to be of satisfactory diagnostic quality. However, the inventors have discovered that the lower x-ray dosage associated with the fluoro mode can provide cardiac angiography images of satisfactory quality for the required clinical decisions when the images are processed using machine learning models trained to segment angiographic images as described above. In an embodiment, the required clinical decisions that are envisioned in this invention pertain to the visualization of the epicardial coronary arteries. These are the larger coronary arteries that run on the surface of the cardiac myocardium before penetration into the actual muscle of the myocardium. The larger vessels are assessed for candidacy for stent placement in the case of significant narrowing (which may be considered to be a reduction in the luminal diameter by 70% or more).

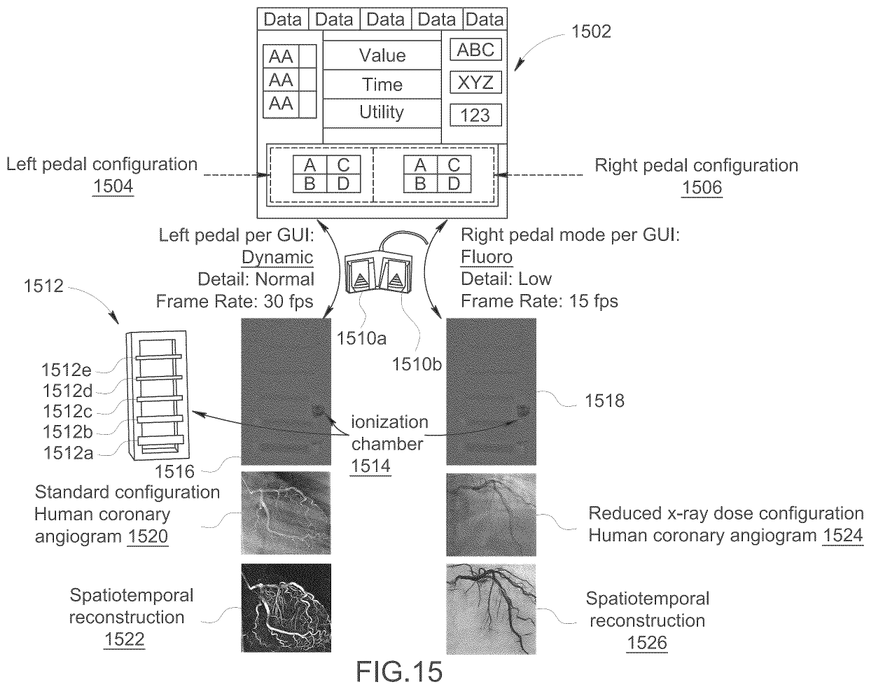

FIG. 15 illustrates a graphical user interface (“GUI”) 1502 for a contemporary angiographic imaging system, such as the General Electric Innova series of angiography products. Similar or analogous GUIs may be generated for other contemporary angiography products such as those made by Siemens, Phillips, or Toshiba. In the GUI 1502, there is a panel on the lower left at 1504 called the left pedal configuration and a panel on the lower right at 1506 called the right pedal configuration. The active configuration is selected by operation of foot pedals 1510a and 1510b connected to the angiographic imaging system and under the control of the angiographer.

In the example shown, there is a left foot pedal 1510a and a right pedal 1510b. When the left foot pedal 1510a is pressed, the left pedal configuration 1504 is selected and applied. For example, when the left foot pedal 1510a is pressed, a dynamic mode may be applied to the system wherein a conventional dosage of x-ray radiation is applied. When the right pedal 1510b is selected, the configuration in the right lower panel 1506 for angiography x-ray dose settings is selected and applied. For example, when the right foot pedal 1510b is pressed, a fluoro mode may be applied wherein a lower dosage of x-ray radiation is applied in comparison to the dynamic mode. The frame rates in the left and right pedal configurations may also be different from one another and selected for application via the foot pedals. For example, the frame rate in the left pedal configuration 1504 may be a conventional frame rate of 30 fps, and the frame rate in the right pedal configuration 1506 may be a reduced frame rate of 15 fps.

As noted above, the inventors have discovered that the fluoro mode in contemporary angiographic imaging systems may be applied in a prescribed way, disclosed here, for generating cardiac angiography images at low x-ray doses, and converting them into images that are satisfactory for the intended clinical use via spatiotemporal reconstruction with a machine learning model (e.g., the machine learning model 1002 in FIG. 10 or the machine learning model 1102 in FIG. 11).

FIG. 15 also illustrates a method for cross-calibration of one angiographic imaging system or machine to another across different brand manufacturers of angiography machines. This cross-brand calibration system includes an in vivo target 1512 that has a sequence of plastic tubes 1512a-e each holding iodinated contrast at different concentrations. The target 1512 also includes on the edge an ionization chamber 1514 that can produce an electrical current that is dependent on the x-ray dosage that hits the ionization chamber. This is a way of internally standardizing x-ray dose that is independent of the settings of the graphical user interface. We find by testing and experience that the settings on the graphical user interface on the various angiography brands do not provide numerical x-ray doses. Therefore, the present technique provides a method for standardizing x-ray doses that arrive at the imaging plate between angiography brands.

FIG. 15 shows two images 1516 and 1518 of the in vitro target 1512 that were obtained in the dynamic and fluoro modes, respectively. That is, image 1516 was obtained with the left pedal dynamic mode and image 1518 was obtained with the lower x-ray dose right pedal fluoro mode. A careful inspection of these images discloses that there is comparatively greater noise in image 1518 from the lower x-ray dose. These in vitro targets 1512 do not offer a satisfactory substrate for the deep learning spatiotemporal reconstruction offered by the machine learning models described herein because the reconstruction technique is based on training with human coronary angiography. The structures of interest in human coronary angiography are curved and branching arteries that move sharply with a heartbeat. These phenomena are not present in the in vitro targets 1512, therefore these do not serve as complete calibration systems for the prescription of a low dose angiography system.

Referring still to FIG. 15, image 1520 is a human coronary angiogram obtained at conventional x-ray and chemical contrast doses. When supplied to the spatiotemporal deep learning reconstruction method described herein, this produces images 1522 in which even microvasculature may be visualized. On the other hand, image 1524 is a human coronary angiogram captured by an angiographical imaging system operating in the fluoro mode. Such an image is not intended for diagnostic permanent record nor for clinical decision making such as the placement of a stent. However, in this particular instance, the inventors provided image 1524 to the spatiotemporal deep learning method described herein. The resulting image 1526 is of sufficient quality to visualize the epicardial coronary arteries and potentially make appropriate clinical decisions concerning stent placement in them. This is not the case with image 1524.

While images 1520 and 1524 represent anonymized data captured from human coronary angiography, it will be appreciated that, for generalized calibration purposes, suitable data may be obtained from animal coronary angiography such as in a pig model. This would offer a greater range of available x-ray dose and chemical contrast doses.

The present invention may include a method, system, device, and/or a computer program product at any possible technical detail level of integration. The computer program product may include a computer readable storage medium (or media) having computer readable program instructions thereon for causing a processor to carry out aspects of the present invention.

Computer readable program instructions described herein can be downloaded to respective computing/processing devices from a computer readable storage medium or to an external computer or external storage device via a network, for example, the Internet, a local area network, a wide area network and/or a wireless network. The network may comprise conductive transmission cables, optical transmission fibers, wireless transmission, routers, firewalls, switches, gateway computers and/or edge servers. A network adapter card or network interface in each computing/processing device may receive computer readable program instructions from the network and forward the computer readable program instructions for storage in a computer readable storage medium within the respective computing/processing device.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a non-transitory computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While example embodiments of a system and method for angiographic dose reduction using machine learning have been disclosed, persons of ordinary skill in the art will appreciate that other embodiments may be derived from the present disclosure without deviating from the spirit and scope of the appended claims.

What is claimed is:

1. A method of training a machine learning system to facilitate real time adjustment of a chemical contrast agent dosage or an x-ray radiation dosage during angiographic imaging, the method comprising:
providing, as an input to a first machine learning model, via a processor:
a first set of one or more angiographic images obtained using a first chemical contrast agent dosage and a first x-ray radiation dosage; and
a concordance metric indicating a degree of similarity between an estimated segmentation of the first set of one or more angiographic images generated by a second machine learning model and one or more segmented benchmark angiographic images provided as inputs to the second machine learning model, wherein the segmented benchmark angiographic images were obtained using a second chemical contrast agent dosage or a second x-ray radiation dosage that is higher than the first chemical contrast agent dosage or the first x-ray radiation dosage;
generating, as an output of the first machine learning model, via a processor, an estimated concordance metric for the first set of one or more angiographic images;
comparing, via a processor, the estimated concordance metric and the inputted concordance metric; and
performing, via a processor, a back propagating step to adjust parameters of the first machine learning model based on the comparing step.

2. The method of claim 1, wherein the concordance metric comprises a loss metric.

3. The method of claim 2, wherein the concordance metric further comprises a loss metric slope.

4. The method of claim 1, wherein the concordance metric comprises a Dice coefficient.

5. The method of claim 4, wherein the concordance metric further comprises a Dice coefficient slope.

6. The method of claim 1, wherein the providing step further comprises providing, as an input to the first machine learning model, via a processor, a dosage value based on the first chemical contrast agent dosage or the first x-ray radiation dosage used to obtain the first set of one or more angiographic images.

7. The method of claim 1, wherein the first and second machine learning models are separate machine learning models.

8. A method of acquiring angiographic images with real time dose adjustment, the method comprising:
obtaining, via an angiographic imaging device, a third set of one or more angiographic images using a third chemical contrast agent dosage and a third x-ray radiation dosage;
providing, via a processor, the third set of one or more angiographic images to a first machine learning model trained according to claim 1;
generating, with the first machine learning model, via the processor, an estimated concordance metric based on the third set of one or more angiographic images.

9. The method of claim 8, further comprising generating, via a processor, a suggested adjustment to the third chemical contrast dosage or the third x-ray radiation dosage based on the generated concordance metric.

10. The method of claim 8, further comprising:
obtaining, via the angiographic imaging device, a fourth set of one or more angiographic images using a fourth chemical contrast dosage and a fourth x-ray radiation dosage based on the generated concordance metric.

11. The method of claim 9, further comprising:
providing, as an input to a second machine learning model, via a processor, the fourth set of one or more angiographic images; and
generating, with the second machine learning model, via a processor, an estimated segmentation of the fourth set of one or more angiographic images.

12. The method of claim 8, further comprising:
adjusting, via a processor, an x-ray radiation dosage emitted by the angiographic imaging device based on the generated concordance metric.

13. The method of claim 8, further comprising:
adjusting, via a processor, a chemical contrast agent dosage administered by an autoinjector based on the generated concordance metric.

14. The method of claim 8, wherein the concordance metric comprises a loss metric coefficient.

15. The method of claim 14, wherein the concordance metric further comprises a loss metric coefficient slope.

16. The method of claim 8, wherein the concordance metric comprises a Dice coefficient.

17. The method of claim 16, wherein the concordance metric further comprises a Dice coefficient slope.

18. The method of claim 8, further comprising comparing the concordance metric with a predetermined value.

19. The method of claim 18, wherein the predetermined value is a minimum acceptable probability of concordance.

20. The method of claim 19, further comprising reducing the chemical contrast agent dosage or the x-ray radiation dosage if the concordance metric is greater than the minimum acceptable probability of concordance.

21. The method of claim 8, wherein the concordance metric is determined for an individual angiographic image frame and used to adjust dosage for a subsequent angiographic image frame.

22. The method of claim 8, wherein the concordance metric is determined for a plurality of angiographic image frames and used to adjust dosage for a subsequent plurality of angiographic image frames.

23. The method of claim 9, further comprising communicating the suggested dosage adjustment to a health care provider.

24. A system for real time adjustment of a chemical contrast agent dosage or an x-ray radiation dosage during angiographic imaging comprising:
one or more processors; and
a memory storing instructions executable by the one or more processors to:
obtain one or more angiographic images acquired using a first chemical contrast agent dosage and a first x-ray radiation dosage;
provide the one or more angiographic images to a first machine learning model trained to generate a concordance metric indicative of a quality of an estimated segmentation of the one or more angiographic images by a second machine learning model trained to segment angiographic images; and
cause the chemical contrast dosage or the x-ray radiation dosage to be adjusted based on the generated concordance metric.

25. The system of claim 24, wherein the concordance metric comprises a loss metric.

26. The system of claim 25, wherein the concordance metric further comprises a loss metric slope.

27. The system of claim 24, wherein the concordance metric comprises a Dice coefficient.

28. The system of claim 27, wherein the concordance metric further comprises a Dice coefficient slope.

29. The system of claim 24, wherein the instructions stored in the memory comprise instructions executable by the one or more processors to compare the concordance metric with a predetermined value.

30. The system of claim 29, wherein the predetermined value is a minimum acceptable probability of concordance.

31. The system of claim 30, wherein the instructions stored in the memory comprise instructions executable by the one or more processors to reduce the chemical contrast agent dosage or the x-ray radiation dosage if the concordance metric is greater than the minimum acceptable probability of concordance.

32. The system of claim 24, wherein the instructions stored in the memory further comprise instructions executable by the one or more processors to obtain a subsequent set of one or more angiographic images using an adjusted chemical contrast dosage or x-ray radiation dosage.

33. The system of claim 32, wherein the instructions stored in the memory further comprise instructions executable by the one or more processors to:
provide, as an input to the second machine learning model, the subsequent set of one or more angiographic images; and
generate, with the second machine learning model, an estimated segmentation of the subsequent set of one or more angiographic images.

34. The system of claim 24, wherein the instructions stored in the memory comprise instructions executable by the one or more processors to communicate a suggested dosage adjustment to a health care provider.

35. A computer program product comprising one or more non-transitory computer readable storage media encoded with instructions that, when executed by one or more processors, cause the one or more processors to:
obtain one or more angiographic images acquired using a first chemical contrast agent dosage and a first x-ray radiation dosage;
provide the one or more angiographic images to a first machine learning model trained to generate a concordance metric indicative of a quality of an estimated segmentation of the one or more angiographic images by a second machine learning model trained to segment angiographic images; and
cause the chemical contrast dosage or the x-ray radiation dosage to be adjusted based on the generated concordance metric.

36. The computer program product of claim 35, wherein the concordance metric comprises a loss metric.

37. The computer program product of claim 36, wherein the concordance metric further comprises a loss metric slope.

38. The computer program product of claim 35, wherein the concordance metric comprises a Dice coefficient.

39. The computer program product of claim 38, wherein the concordance metric further comprises a Dice coefficient slope.

40. The computer program product of claim 35, wherein the instructions comprise instructions executable by one or more processors to compare the concordance metric with a predetermined value.

41. The computer program product of claim 40, wherein the predetermined value is a minimum acceptable probability of concordance.

42. The computer program product of claim 41, wherein the instructions comprise instructions executable by one or more processors to reduce the chemical contrast agent dosage or the x-ray radiation dosage if the concordance metric is greater than the minimum acceptable probability of concordance.

43. The computer program product of claim 35, wherein the instructions comprise instructions executable by one or more processors to obtain a subsequent set of one or more angiographic images using an adjusted chemical contrast dosage or x-ray radiation dosage.

44. The computer program product of claim 43, wherein the instructions comprise instructions executable by one or more processors to:
provide, as an input to the second machine learning model, the subsequent set of one or more angiographic images; and
generate, with the second machine learning model, an estimated segmentation of the subsequent set of one or more angiographic images.

45. The computer program product of claim 35, wherein the instructions comprise instructions executable by one or more processors to communicate a suggested dosage adjustment to a health care provider.
